(19) United States
(12) Patent Application Publication
(10) Pub. No.: US 2017/0284975 A1
(43) Pub. Date: Oct. 5, 2017

(54) METHODS TO DETERMINE THE DISTRIBUTION PROFILES OF CIRCULATING MICRORNAS

(71) Applicant: The Regents of the University of California, Oakland, CA (US)

(72) Inventors: Wenwan Zhong, Riverside, CA (US); Jonathan Ashby, Riverside, CA (US); Kenneth Flack, Riverside, CA (US)

(73) Assignee: The Regents of the University of California, Oakland, CA (US)

(21) Appl. No.: 15/507,159

(22) PCT Filed: Sep. 3, 2015
(86) PCT No.: PCT/US2015/048341

§ 371 Date: Feb. 27, 2017

Related U.S. Application Data
(60) Provisional application No. 62/045,503, filed on Sep. 3, 2014.

(51) Int. Cl. G01N 30/00; C12Q 1/68

(52) U.S. Cl. CPC G01N 30/0005; C12Q 1/68; G01N 2030/003; C12N 2310/141

(57) ABSTRACT

The disclosure provides methods for rapid fractionation of circulating RNAs based on the type of carriers they locate in. The disclosure further provides that the methods of the disclosure can be used for diagnosing a disorder in a subject by identifying specific microRNA biomarkers associated with that disorder.

### CROSS REFERENCE TO RELATED APPLICATIONS

This application claims priority under 35 U.S.C. §119 from Provisional Application Ser. No. 62/045,503, filed Sep. 3, 2014, the disclosure of which is incorporated herein by reference.

### GOVERNMENT LICENSE RIGHTS

This invention was made with Government support under Grant Nos. CHE-1057113 and DGE-0813967, awarded by National Science Foundation. The Government has certain rights in the invention.

### TECHNICAL FIELD

The disclosure provides methods for rapid fractionation of circulating microRNAs, viral RNA and long-non-coding RNA (lncRNA) based on their associated carrier molecules. The disclosure further provides that the methods of the disclosure can be used for diagnosing a disorder in a subject by identifying specific microRNA, lncRNA and viral RNA makers associated with that disorder and specific carriers.

### BACKGROUND

Circulating microRNAs have been thought to be good biomarkers for disease diagnosis, because they could be specifically secreted by diseased cells, such as cancer cells. Considering the key roles of microRNAs in regulating gene expression, the active secretion of microRNAs could be highly relevant to disease development.

Conventionally, total microRNAs are extracted from patient's serum, and the expression profiles are analyzed to see whether the patterns can be used to indicate disease stage. But such patterns have not revealed very convincing miRNA markers, although a lot of screenings have been done.

### SUMMARY

The disclosure provides a fractionation method for determining the distribution of circulating RNAs in a sample, comprising fractionating a biological fluid sample obtained from a subject into fractions comprising at least an exosome fraction, protein fraction and lipoprotein fraction, wherein each fraction comprises RNA carriers; and determining or quantitating the RNAs in each of the fractions to generate a distribution profile for the RNAs to RNA carriers in the sample. In one embodiment, the fractionating is by performing asymmetrical flow field-flow fractionation (AF4) and collecting a plurality of eluents. In another embodiment, the fractionating is by a chip-based microfluidics system. In still another embodiment of any of the foregoing the biological fluid sample is a serum sample. In a further embodiment, a serum sample is fractionated using a trapezoidal separation channel about 0.350 mm in thickness and a tip-to-tip length of about 275 mm, with an inlet triangle width of about 20 mm and outlet width of about 5 mm. In yet a further embodiment, the surface area of the accumulation wall is about 3160 mm² with a molecular weight cutoff value of 10 kDA. In still a further embodiment, the plurality of eluents are collected as 1 minute eluents over a period of 20 to 25 minutes. In another embodiment, at least six fractions of the biological fluid sample are generated from the plurality of eluents. In a further embodiment, the six fractions result from combining 1 minute eluents collected over six separate and non-overlapping time periods. In a further embodiment, each of the six factions are enriched with an RNA carrier protein of a specific hydrodynamic diameter. In another embodiment, the fractions are enriched with proteins, high density lipoprotein (HDL), low density lipoprotein (LDL) and exosome. In still another embodiment, the RNAs are determined or quantified by deep sequencing or RT-qPCR. In another embodiment of any of the foregoing embodiments, the RNAs include microRNAs or lncRNAs, or viral RNAs. In a further embodiment, the microRNAs or lncRNAs or viral RNAs are biomarkers associated with a disease or disorder. In still a further embodiment, the disorder is cancer. In yet a further embodiment, the cancer is breast cancer. In one embodiment, of any of the foregoing, the RNAs are microRNAs comprising the sequence of SEQ ID NO:1, 2, 3, 4, 5, 6, 7, 8, and/or 9. In another embodiment, the RNAs are selected from the group consisting of let-7a, let-7b, let-7c, let-7d, let-7e, let-7f, let-7g, let-7i, miR-1, miR-100, miR-101, miR-103, miR-105, miR-106a, miR-106b, miR-107, miR-10a, miR-10b, miR-122a, miR-124a, miR-125a, miR-125b, miR-126, miR-126*, miR-127, miR-128a, miR-128b, miR-129, miR-130a, miR-130b, miR-132, miR-133a, miR-133b, miR-134, miR-135a, miR-135b, miR-136, miR-137, miR-138, miR-139, miR-140, miR-141, miR-142-3p, miR-142-5p, miR-143, miR-144, miR-145, miR-146a, miR-146b, miR-147, miR-148a, miR-148b, miR-149, miR-150, miR-151, miR-152, miR-153, miR-154, miR-154*, miR-155, miR-15a, miR-15b, miR-16, miR-17-3p, miR-17-5p, miR-181a, miR-181b, miR-181c, miR-181d, miR-182, miR-182*, miR-183, miR-184, miR-185, miR-186, miR-187, miR-188, miR-189, miR-18a, miR-18a*, miR-18b, miR-190, miR-191, miR-191*, miR-192, miR-193a, miR-193b, miR-194, miR-195, miR-196a, miR-196b, miR-197, miR-198, miR-199a, miR-199a*, miR-199b, miR-19a, miR-19b, miR-200a, miR-200a*, miR-200b, miR-200c, miR-202, miR-202*, miR-203, miR-204, miR-205, miR-206, miR-208, miR-20a, miR-20b, miR-21, miR-210, miR-211, miR-212, miR-213, miR-214, miR-215, miR-216, miR-217, miR-218, miR-219, miR-22, miR-220, miR-221, miR-222, miR-223, miR-224, miR-23a, miR-23b, miR-24, miR-25, miR-26a, miR-26b, miR-27a, miR-27b, miR-28, miR-296, miR-299-3p, miR-299-5p, miR-29a, miR-29b, miR-29c, miR-301, miR-302a, miR-302a*, miR-302b, miR-302b*, miR-302c, miR-302c*, miR-302d, miR-30a-3p, miR-30a-5p, miR-30b, miR-30c, miR-30d, miR-30e-3p, miR-30e-5p, miR-31, miR-32, miR-320, miR-323, miR-324-3p, miR-324-5p, miR-325, miR-326, miR-328, miR-329, miR-33, miR-330, miR-331, miR-335, miR-337, miR-338, miR-339, miR-33b, miR-340, miR-342, miR-345, miR-346, miR-34a, miR-34b, miR-34c, miR-361, miR-362, miR-363, miR-363*, miR-365, miR-367, miR-368, miR-369-3p, miR-369-5p, miR-370, miR-371, miR-372, miR-373, miR-373*, miR-374, miR-375, miR-376a, miR-376a*, miR-376b, miR-377, miR-378, miR-379, miR-380-3p, miR-380-5p, miR-381, miR-382, miR-383, miR-384, miR-409-3p, miR-409-5p, miR-410, miR-411, miR-412, miR-421, miR-422a, miR-422b, miR-423, miR-424, miR-425, miR-425-5p, miR-429, miR-431, miR-432, miR-432*, miR-433, miR-448, miR-449, miR-450, miR-451, miR-452, miR-452*, miR-453, miR-455, miR-483, miR-484, miR-485-3p, miR-485-5p, miR-486, miR-487a, miR-487b, miR-488, miR-489, miR-490, miR-491, miR-492, miR-493, miR-493-3p, miR-494, miR-495, miR-496, miR-497, miR-498, miR-499, miR-500, miR-501, miR-502, miR-503, miR-504, miR-505, miR-506, miR-507, miR-508, miR-509, miR-510, miR-511, miR-512-3p, miR-512-5p, miR-513, miR-514, miR-515-3p, miR-515-5p, miR-516-3p, miR-516-5p, miR-517*, miR-517a, miR-517b, miR-517c, miR-518a, miR-518a-2*, miR-518b, miR-518c, miR-518c*, miR-518d, miR-518e, miR-518f, miR-518f*, miR-519a, miR-519b, miR-519c, miR-519d, miR-519e, miR-519e*, miR-520a, miR-520a*, miR-520b, miR-520c, miR-520d, miR-520d*, miR-520e, miR-520f, miR-520g, miR-520h, miR-521, miR-522, miR-523, miR-524, miR-524*, miR-525, miR-525*, miR-526a, miR-526b, miR-526b*, miR-526c, miR-527, miR-532, miR-542-3p, miR-542-5p, miR-544, miR-545, miR-548a, miR-548b, miR-548c, miR-548d, miR-549, miR-550, miR-551a, miR-552, miR-553, miR-554, miR-555, miR-556, miR-557, miR-558, miR-559, miR-560, miR-561, miR-562, miR-563, miR-564, miR-565, miR-566, miR-567, miR-568, miR-569, miR-570, miR-571, miR-572, miR-573, miR-574, miR-575, miR-576, miR-577, miR-578, miR-579, miR-580, miR-581, miR-582, miR-583, miR-584, miR-585, miR-586, miR-587, miR-588, miR-589, miR-590, miR-591, miR-592, miR-593, miR-594, miR-595, miR-596, miR-597, miR-598, miR-599, miR-600, miR-601, miR-602, miR-603, miR-604, miR-605, miR-606, miR-607, miR-608, miR-609, miR-610, miR-611, miR-612, miR-613, miR-614, miR-615, miR-616, miR-617, miR-618, miR-619, miR-620, miR-621, miR-622, miR-623, miR-624, miR-625, miR-626, miR-627, miR-628, miR-629, miR-630, miR-631, miR-632, miR-633, miR-634, miR-635, miR-636, miR-637, miR-638, miR-639, miR-640, miR-641, miR-642, miR-643, miR-644, miR-645, miR-646, miR-647, miR-648, miR-649, miR-650, miR-651, miR-652, miR-653, miR-654, miR-655, miR-656, miR-657, miR-658, miR-659, miR-660, miR-661, miR-662, miR-663, miR-7, miR-9, miR-9*, miR-92, miR-93, miR-95, miR-96, miR-98, miR-99a, miR-99b and any combination thereof. In another embodiment, the chip-based microfluidic system comprises a microfluidic chip comprising at least 3 channels; at least 3 reservoirs; and a sample reservoir, wherein the channels fluidly connect the at least 3 reservoirs and sample reservoir; a first bead reagent comprising magnetic beads and an antibody that interacts with an antigen on exosomes; and a second bead reagent comprising cationically charged beads. In a further embodiment, the antibody is an anti-CD63 antibody. In a further embodiment, the method comprises (i) adding serum to the sample reservoir; (a) adding the first bead reagent to the sample reservoir; applying a magnetic field to the sample reservoir and moving the first bead reagent with the magnetic field through a first channel of the at least 3 channels to a first reservoir of the at least 3 reservoirs; disrupting the exosomes in the first reservoir; removing the first bead reagent; adding a second bead reagent to the first reservoir; (b) adding GuHCl, KCl, and a detergent to the sample reservoir to dissociate RNA from proteins; add the second bead reagent to the sample reservoir to bind RNA; moving the second bead reagent through a second channel of the at least 3 channels to a second reservoir of the at least 3 reservoirs; and (c) adding guanidine thiocyanate, a detergent, and ethanol to the sample reservoir to dissociate RNA from lipoproteins; add the second bead reagent to the sample reservoir to bind RNA; moving the second bead reagent through a third channel of the at least 3 channels to a third reservoir of the at least 3 reservoirs, (ii) extracting RNA from each of the first, second and third reservoir. In a further embodiment, the method further comprises reagents that can destroy the protein-RNA interaction, or the lipoprotein complexes. In a further embodiment, the reagents are a mixture of surfactant, organic solvent, chaotropic salts.

The disclosure also provides a method for diagnosing whether a subject has a disorder, comprising comparing the distribution of circulating RNAs obtained by using the method of any of the foregoing embodiments between a healthy subject(s) and subject(s) with the disorder, wherein a difference identifies a risk of the disease or disorder.

The disclosure also provides a kit for carrying out any of the methods described herein, wherein the kit is compartmentalized to contain reagents and devices for performing the methods. In a further embodiment, the kit comprises a microfluidic device, a first bead reagent, a second bead reagent, and reagents that can destroy the protein-RNA interaction, or can destroy the lipoprotein complexes.

The disclosure provides methods for the rapid fractionation of circulating microRNAs (miRNAs) based on the type of associated carrier. The fractionated miRNAs are collected, identified, and quantified by RT-qPCR. A distribution profile of each of the targeted miRNAs is then obtained. The methods disclosed herein feature rapid fractionation, high recovery, and have a low possibility of disrupting the binding between miRNAs and their carriers. Further, the methods of the disclosure enable comprehensive profiling of the location of miRNAs in various carriers, which can reveal the more sensitive and specific microRNA markers for disorder diagnosis. The distribution profile contains much richer information for interpreting the secretion and transportation pathway of the microRNAs, and their roles in disease development. Comparison of the distribution profiles of circulating miRNAs collected from healthy subject(s) and from patient(s) with a disorder(s) can not only reveal which miRNAs are associated with the disorder but can also indicate the stage of the disorder based upon which carrier is associated with the miRNA.

In a particular embodiment, the disclosure provides a rapid fractionation method for determining the distribution of circulating miRNAs in a sample, comprising: fractionating a serum sample obtained from a subject, by performing asymmetrical flow field-flow fractionation (AF4) on the sample and collecting a plurality of eluents; combining the plurality of eluents into fractions, wherein each fraction is enriched with a different miRNA carrier; quantitating the level of a set of miRNAs in each of the collected fractions to generate distribution profiles for the miRNAs to carriers in the sample; and determining the distribution of circulating miRNAs in the sample. In a further embodiment, the serum sample is fractionated using a trapezoidal separation channel about 0.350 mm in thickness and a tip-to-tip length of about 275 mm, with an inlet triangle width of about 20 mm and outlet width of about 5 mm. In yet a further embodiment, the surface area of the AF4 accumulation wall is about 3160 mm² with a molecular weight cutoff value of 10 kDA. In another embodiment, the plurality of eluents are collected as 1 minute eluents over a period of 20 to 25 minutes. In yet another embodiment, at least six fractions of the serum sample is generated from the plurality of eluents. In a further embodiment, the six fractions result from combining 1 minute eluents collected over six separate and non-overlapping time periods. In yet a further embodiment, each of the six factions is enriched with a miRNA carrier protein of a specific hydrodynamic diameter.

In a certain embodiment, a method of the disclosure comprises fractions that are enriched with a miRNA carrier protein selected from high density lipoprotein (HDL), low density lipoprotein (LDL), and exosome. In another embodiment, a method disclosed herein comprises quantifying miRNAs by using RT-qPCR.

In a particular embodiment, a method of the disclosure comprises a set of miRNAs that are biomarkers associated with a disorder, such as a cancer, diabetes, obesity, epilepsy, liver disease (e.g., NASH or NAFLD), coronary artery disease, Alzheimer Disease, polycystic ovary syndrome, endometriosis, kidney disease (e.g., minimal change disease, focal segmental glomerulosclerosis). In a further embodiment, a method of the disclosure comprises a set of microRNAs that are biomarkers associated with breast cancer, such as those microRNAs comprising the sequence of SEQ ID NO:1, 2, 3, 4, 5, 6, 7, 8, and/or 9.

In a certain embodiment, a method disclosed herein can be used to diagnose whether a subject has a disorder, comprising: comparing the distribution of circulating microRNAs obtained by using a method of the disclosure with the distribution of circulating microRNAs from a healthy subject(s) and/or subject(s) with the disorder obtained by using that same method.

### DESCRIPTION OF DRAWINGS

FIG. 1A-E provides for the optimization of AF4 flow profile using exosome isolates. (A) Constant flow rates of 3.0 mL/min cross and 0.3 mL/min detector flow. (B) Post-AF4 collection (cross-flow turned off). (C-E) Rampdown of cross-flow from 3.0 mL/min to zero cross flow over 30 minutes (C), 20 minutes (D), and 15 minutes (E). Absorbance detection for all samples was measured at 280 nm. All isolates were prepared from healthy human male pooled serum. (F) depicts a graph showing miRNA levels corresponding to various fractions, each fraction associated with a different RNA carrier or set of carriers, fractions F1-F6 correspond to columns from left to right for each miRNA marker.

FIG. 2A-B presents (A) AF4 of protein and nanoparticle standards; and (B) AF4 of exosome isolates and lipoprotein complex standards. All samples were detected via absorbance at 280 nm. Exosome isolates were prepared from healthy human male pooled serum.

FIG. 3 demonstrates that the addition of a 5-minute constant flow region at the start of the AF4 separation allows for improved resolution of analytes in the exosome isolate. The AF4 flow profile for this method included 5 minutes with a cross-flow of 3.0 mL/min and 0.3 mL/min detector flow, followed by a 15 minute rampdown of the cross-flow from 3.0 mL/min to zero flow. Absorbance detection was conducted at 280 nm.

FIG. 4A-B presents (A) fractograms (UV absorption at 280 nm) for serum before and after spiked with HDL or LDL; and (B) comparison of fractograms (detected by fluorescence with 480ex/510em) of serum and exosome extract after DiO staining.

FIG. 5 presents fractograms for serum samples from healthy individuals (controls) and BC patients (cases). The table shows the time range of each collected fraction; and the RSD values of the peak elution time for each fraction. N/A means no distinct peak in the fraction.

FIG. 6A-D provides (A) absorbance and (B) DiO-stained fluorescence fractograms of healthy serum samples. Black—Control 1, Red—Control 2. (C) Absorbance and (D) DiO-stained fluorescence fractograms of serum samples from BC patients. Black—Case #1, Red—Case #2. All absorbance measurements were taken at 280 nm. All fluorescence fractograms were measured at an excitation of 485 nm and an emission of 510 nm. Samples were fractionated using the optimized AF4 fractionation protocol.

FIG. 7A-B provides (A) spectral counting results for selected lipoproteins in the AF4 fractions; and (B) ELISA detection of CD-63 in the collected fractions.

FIG. 8 presents the recovery of hsa-miR-16 from pure serum or AF4 fractions.

FIG. 9A-C presents (A) the distribution profiles of the 8 tested miRNAs in the serum collected from one breast cancer patient (Case #1); (B) change in the averaged Log value of miRNA copies (counting all four tests—2 samples with 2 repeats-in each group) between the controls and cases. “*” marked out those showing significant difference between healthy donors (controls) and BC patients (cases) with p<0.05; and (C) the score plot of principle component 1 vs. principle component 2 obtained by PCA on the miRNA quantity of miR-16, -17, -375, and -122 in certain fractions as indicated in the text. The arbitrary circles illustrated the separation between the control and case groups.

FIG. 10A-C provides RT-qPCR analysis of each sample for each fraction. (A) control 1, (B) control 2, and (C) case 2. The calculated number of copies for each is normalized based on the number of copies of cel-mir-67 present in each sample. The Y-axis is the Log value of the copy number of the miRNA.

FIG. 11 shows a schematic illustration of the overall design for on-chip miRNA distribution profiling technique.

FIG. 12 shows an exemplary microfluidic device for use in the methods and systems of the disclosure. A total of 3 channels are depicted, each dedicated to one type of carriers. A first channel is used for extraction of protein-bound RNAs, a second channel for lipoprotein-associated RNA, and a third channel for exosomal RNAs. In order to prevent unwanted and non-specific adsorption of either RNA or serum components, the device surfaces can be modified with octamethyl siloxane to have high hydrophobicity and inertness.

FIG. 13 shows a method of making a microfluidic device of the disclosure.

FIG. 14 shows examples of beads and their design for use in obtaining an exosome fraction from a sample.

FIG. 15 shows examples of beads and their design for use in obtaining protein and lipoprotein fractions.

FIG. 16A-D shows various separation traces. (A) AF4 separation traces (fractograms) collected by the fluorescence detector for analysis of exosomes isolated by the immuno-beads as done in our microchip profiling technique (dotted line), and by the Invitrogen kit (solid line). (B) Top: Fractions collected during serum separation by AF4 (detection was done by UV absorption). The eluents collected were dried and the proteins were collected for CD 63 quantification by ELISA, and result was shown in the bottom bar plot. The quantity was the average of three repeated measurements and the error bars were the standard deviations. (C) Comparison of the CD63 concentration in exosomes prepared by the immuno-beads isolation method, and the Invitrogen kit. (D) Fractograms for exosomes isolated by the immuno-beads before (solid) and after (square dot) treatment with the disruption solution. Fluorescence detection was done by DiO staining and fluorescence with 480ex/510em.

FIG. 17 shows fractograms for the exosome-depleted serum before (solid) and after treatment with the protein disruption solution (square dot) and by the lipoprotein disruption solution (dash dot). The solid block highlights the position where the HDL standard would be eluted, and the square dot block indicates the elution window for the LDL standard. Fluorescence detection was done by DiO staining and fluorescence with 480ex/510em.

FIG. 18 shows a comparison of percent recovery of the spiked miRNA in serum using a bead-based extraction method of the disclosure and the commercial kits, including the TRIzol LS reagent with different durations, the GeneJet RNA purification kit, and the PureLink RNA kit, all distributed by Thermo Fisher.

FIG. 19A-C shows a comparison of the miRNA copies obtained from the on-chip and AF4-based distribution profiling methods, as well as from immuno-capture using the antibody-conjugated magnetic beads. Four miRNAs were selected in the comparison. (A) The protein-bound miRNAs recovered from Channel 1 on the microchip and in Fraction 1 from AF4 separation of the healthy serum purchased from Sigma. (B) The exosomal miRNAs recovered from Channel 3 on the microchip, by the Invitrogen Total Exosome Isolation kit, and in Fraction 6 from AF4 separation. The exosomes from AF4 and from Invitrogen kit were treated with the TRIzol LS reagent. (C) The lipoprotein-associated miRNAs obtained in Channel 2 on the microchip, adding up from fraction 2-5 in AF4 separation, and with immuno-beads conjugated to anti-HDL/LDL IgGs.

FIG. 20A-D shows distribution profiles of the sera collected one patient (A) and one healthy individual (B). (C) The ratio of the average miRNA content in all 7 cases over the average value from 3 controls found in all three fractions (white, grey, and black bars), compared with that found in the total miRNA content of all fractions (patterned). (D) The score plot of PC1 vs. PC2 for all samples. The cases were shown as black circles, and the controls were red triangles.

### DETAILED DESCRIPTION

As used herein and in the appended claims, the singular forms “a,” “and,” and “the” include plural referents unless the context clearly dictates otherwise. Thus, for example, reference to “a fraction” includes a plurality of such fractions and reference to “the miRNA” includes reference to one or more miRNAs and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies that might be used in connection with the description herein. With respect to any term that is presented in the one or more publications that is similar to, or identical with, a term that has been expressly defined in this disclosure, the definition of the term as expressly provided in this disclosure will control in all respects.

Cells communicate with their surrounding environment via many different pathways, including cell-cell interactions, cell-matrix interactions, hormones, growth factors, cytokines, hormones and the like. Long range effects between cells can be performed through a process of secreting factors that travel through the blood stream to act upon a distant cells. More recently, evidence shows the vesicles such as exosomes are capable of mediating such communications.

Early detection of cancer can enhance the survival rate of patients but the success strongly relies on the availability of specific and sensitive biomarkers. One class of promising biomarkers for cancer diagnosis are the microRNAs (miRNAs) and long non-coding RNAs (lncRNAs). miRNAs bind to target mRNAs and inhibit translation or induce degradation of target transcripts. Overexpression of miRNAs that inhibit the tumor suppressor genes can interfere with the anti-oncogenic pathway; while deletion or epigenetic silencing of miRNAs that target oncogenes can increase oncogenic potency. It is also recognized that miRNA profiles more accurately reflect the developmental lineage and tissue origin of human cancers than mRNA profiles. Compared to proteins, miRNAs have simpler structures and less complex post-synthesis processing; and can be detected by the highly sensitive PCR methods. More appealing, miRNAs can be released into the circulation system and be stably present at levels detectible by sensitive techniques like RT-PCR. Accumulating evidence shows that circulating miRNAs exhibit varied patterns between cancer patients and healthy controls, with the patterns of some secretory miRNAs altered in the early stage of cancer initiation. Since sampling from circulating body fluids, like blood, urine, saliva, etc. is considered to be convenient and non-invasive compared to other biopsy methods, more and more research efforts have been devoted to obtaining the comprehensive profiles of circulating miRNAs, and validate their utility as biomarkers.

The microRNAs are bound to certain carriers, such as proteins, lipoprotein particles (like HDL), and exosomes (membranous vesicles with diameter around 30-100 nm, released by cells). The carriers are highly relevant to how the microRNAs are secreted and transported in the circulation system. Therefore, RNAs in particular carriers, but not the sum quantity, are directly related to disease development. Moreover, current methods for fractionating circulating RNAs bound to carriers in serum or plasma are exclusively based upon size exclusion chromatography or ultracentrifugation.

RNA interference (RNAi) is a biological process for the control of gene expression and activity. Recently, RNAi molecules (e.g., miRNA) have been reported to be present in exosomes, high- and low-density lipoproteins (Vickers et al, 2011) (HDL/LDL), large extracellular vesicles, termed microvesicles, and are associated with Argonaut 2 (AGO2) (Arroyo et al., 201 1; Li et al., 2012; Turchinovich et al., 2011).

miRNAs are small non-coding RNAs of 18-24 nucleotides (nt) in length that control gene expression post-transcriptionally. They are synthesized via sequential actions of Drosha and Dicer endonucleases and loaded into the RISC (RNA induced silencing complex) to target mRNAs (Bartel, 2009; Maniataki and Mourelatos, 2005).

miRNAs operate via sequence-specific interaction and pairing of the miRNA-associated RISC (composed of Dicer, TRBP and AG02 proteins) with the target mRNAs (Bartel, 2009). This action inhibits translation and/or causes mRNA destabilization (Filipowicz, 2005). The degree of complementarity of the miRNA and its mRNA target dictates the process of mRNA silencing, either via mRNA destabilization/degradation or by inhibition of translation (Ambros, 2004; Bartel, 2009). If complete complementation is encountered between the miRNA and target mRNA sequence, the RISC complex acts to cleave the bound mRNA for degradation (Ambros, 2004; Bartel, 2009). If absolute complementation is not encountered, as in most cases of miRNAs in animal cells, translation is prevented to achieve gene silencing (Ambros, 2004; Bartel, 2009).

To achieve efficient miRNA-mediated gene silencing, the miRNA must be complexed with the RLC (RISC-loading complex) proteins Dicer, TRBP and AGO2. Within the RLC, Dicer and TRBP are required to process precursor miRNAs (pre-miRNAs), after they emerge from the nucleus via exportin-5, to generate miRNAs and associate with AG02. AG02 bound to the mature miRNA constitutes the minimal RISC and may subsequently dissociate from Dicer and TRBP. Single-stranded miRNAs by themselves incorporate into RISC very poorly and therefore cannot be efficiently directed to its target mRNA for post-transcriptional regulation.

Exosomes are released by cells in vivo and in vitro. By the term “exosome” is meant a lipid-based microparticle or nanoparticle present in a sample (e.g., a biological fluid) obtained from a subject. The term exosome is also referred to in the art as a microvesicle, nanovesicle or extracellular vesicles. In some embodiments, an exosome is between about 20 nm to about 90 nm in diameter. Exosomes are secreted or shed from a variety of different mammalian cell types. Exosomes are small membrane-bound vesicles that carry biological macromolecules from the site of production to target sites either in the microenvironment or at distant sites away from the origin. The content of exosomal content varies with the cell type that produces them as well as environmental factors that alter the normal state of the cell such as viral infection. Exosomes have been shown to contain viral RNA, viral proteins, viral miRNA, cellular miRNA and the like (Singh et al., Viruses, 7(6):3204-25, 2015; Hubert et al., Future Virol., 10(4):357-370, 2015).

Long noncoding RNAs (lncRNAs) include RNA molecules greater than 200 nucleotides in length that have low protein-coding potential. Traditionally viewed as transcriptional noise, they are now emerging as important regulators of cellular functions such as protein synthesis, RNA maturation/transport, chromatin remodeling, and transcriptional activation and/or repression programs. They have been shown to influence biological processes such as stem cell pluripotency, cell cycle, and DNA damage response. Indicative of their important regulatory functions, aberrant expression and function of some lncRNAs have been observed in several types of cancers (see, e.g., U.S. Pat. Publ. No. 2013/0178428, the disclosure of which is incorporated herein by reference).

Circulating microRNAs (miRNAs) are potential biomarkers useful in cancer, diabetes, obesity, epilepsy, liver disease (e.g., NASH or NAFLD), coronary artery disease, Alzheimer Disease, polycystic ovary syndrome, endometriosis, and kidney disease (e.g., minimal change disease, focal segmental glomerulosclerosis) diagnosis. Similarly, long-non-coding RNA (lncRNA) molecules have been associated with various disease as having an effect on gene expression. These RNA molecules have been found to be bound to various carriers such as proteins, lipoprotein particles, and exosomes. It is likely that the miRNAs and lncRNA associated with particular carriers, but not the overall quantity, are related to the disease states (e.g., cancer, cardiovascular, kidney, endometriosis etc.).

One obstacle to using circulating miRNAs as a diagnostic is that not all circulating miRNAs are related to cancer development or disease. The cancer/disease-irrelevant miRNAs can be secreted by blood cells; or be shed after cells die. They could then contribute to large variances in miRNA abundances between individuals and subsidize signals from the cancer-related miRNAs during quantification. It has been known that, the cell-free miRNAs are protected from nucleases in extracellular environments and in body fluids by various types of carriers. The carriers can be proteins like Argonaute (AGO) 2 and GW182 that belong to the RNA-induced silencing complex (RISC); lipoprotein (high-density lipoprotein (HDL) and low density lipoprotein (LDL)) particles that could mediate intracellular communication; or vesicles like the exosomes, which are believed to be one of the exportation routes for miRNAs from malignant cells. While active miRNA secretion by malignant cells could be the consequence of dysregulation of cellular pathways, for-purpose exportation and uptake could be related to tumor progression and metastasis. Therefore, to better eliminate the cancer-irrelevant miRNAs and reveal the more specific miRNA markers, isolation of miRNAs from carriers that are specifically secreted by cancer cells could provide a solution. Thus, HDL and exosomes have recently been focused in study of circulating miRNAs.

Furthermore, viral RNA (vRNA) associated with various carriers can be used to determine the presence of an infection, viral load, or the state of the infection (e.g., active or latent).

As used herein an “RNA carrier” refers to a macromolecule present in a fluid or tissue of a subject and to which RNA is bound or carried in the subject. In one embodiment, the RNA carrier is not a cell (“non-cellular”) (e.g., not a stem cell, parenchymal or other cell). By “bound” means covalently or non-covalently associated with the RNA carrier (e.g., encapsulated in an exosome, linked by H-bonds or other charge association and the like). Examples of RNA carriers include proteins (e.g., Argonaute (AGO)2 and GW182 that belong to the RISC complex), lipids, lipoproteins (e.g., high-density lipoproteins (HDLs) and/or low density lipoproteins (LDLs)), extracellular vesicles (e.g., exosomes), and the like. The term “RNAs” as used herein refers to one or more of miRNA, lncRNA, and viral RNA.

By the term “sample” or “biological sample” is meant any biological fluid obtained from a mammalian subject (e.g., composition containing blood, plasma, urine, saliva, breast milk, tears, vaginal discharge, or amniotic fluid).

While miRNAs enclosed in exosomes, may provide disease state information, the methods disclosed herein have found RNAs bound to other carriers are also highly relevant to disease development, as different carriers are secreted by different pathways and transported to different locations. The actual distribution pattern of RNAs among various carriers is therefore indicative to the stage of a disease and disease diagnosis. By using the methods of the disclosure, RNA quantities in separate carriers can be analyzed, allowing for the identification of specific microRNA, lncRNA and vRNA disease states.

Pure HDL or exosomes are often obtained by ultracentrifugation and immunoaffinity capture. Ultracentrifugation can provide good size/density resolution; but it requires large sample volumes, is very tedious and time-consuming, and typically provides low recovery. Immunoaffinity capture is easy to perform and provides high specificity, but can only target one type of carrier at a time. In one study of miRNA carriers, serum was fractionated with size exclusion chromatography (SEC) to reveal the existence of exosomal and exosome-free circulating miRNAs. In another study, SEC was used to further characterize the HDL isolated by ultracentrifugation. However, in SEC, good separation resolution can only be achieved within a small size range; interaction of biomolecules with the column materials is problematic; and integrity of biocomplexes or vesicle structures after passing through the packed column is questionable.

While recovering RNAs from either pure HDL or exosomes could possibly remove the cancer/disease-irrelevant RNAs shed by normal cells, it is actually not conclusive about which carriers are more important in cancer and disease diagnosis. Thus, study of RNA distribution among all types of carriers is important in answering this question.

The disclosure provides a method for rapid separation of different RNA carriers in a fluid (e.g., serum) from a subject using (i) asymmetrical flow field flow fractionation (AF4) (or an improvement thereof, see, e.g., U.S. Pat. Publ. No. 2009/0301942, which is incorporated herein by reference) or (b) a bead-based microfluidic/chip-based methods. Compared to SEC and ultracentrifugation, asymmetrical flow field-flow fractionation (AF4) and the bead-based microfluidic method are gentler and thus provide for better preservation of the binding between RNAs and their carriers. Due to its non-interactive separation ability, AF4 and the bead-based microfluidic method can be used to isolate intact macromolecular complexes of protein-RNA, lipoprotein-RNA and exosomes containing RNA.

The A4F apparatus and variants thereof are described in various publication including Giddings et al. (Science, 260:1456-1465, 1993) and Carl-Gustav Wahlund et al. (“Properties of an asymmetrical flow field-flow fractionation channel having one permeable wall,” Analytical Chemistry 59, 1332-39, 1987).

Generally an A4F unit includes (1) a bottom assembly structure holding a liquid-permeable frit, usually made from sintered stainless steel particles, (2) a permeable membrane that lies over the frit, (3) a spacer of thickness from about 75 to 800 μm containing a cavity, and (4) a top assembly structure generally holding a transparent plate of material such as glass. The resulting sandwich is held together with screws, bolts, glue or some other means. A generally rectangular or coffin-shaped cavity in the spacer serves as the channel in which separation will occur. The top assembly structure typically contains three holes that pass through the top plate, referred to as ports, that are centered above the channel and permit the attachments of fittings thereto. These ports are: (a) a mobile phase inlet port located near the beginning of the channel and through which is pumped the carrier liquid (the “mobile phase”), (b) a sample port, very close to and downstream of the inlet port, into which an aliquot of the sample to be separated is introduced to the channel, and (c) an exit port through which the fractionated aliquot leaves the channel, downstream from the inlet port and sample port.

A4F channels are used to separate particles including serum proteins, lipids and the like and spanning a size range from a few nanometers to tens of micrometers. The separation of a sample aliquot comprised of such particles depends in turn on the length, breadth, and thickness of the rectangular or coffin-shaped cavity. In addition, it depends on the channel flow rate, the ratio of the cross flow to channel flow, temperature, liquid viscosity, pH, ionicity, the physical composition of the particles themselves, and the type of permeable membrane lying over the frit. By suitably programming the time variation of the channel-to-cross flow ratio, separations of different particle classes may be improved significantly and often a great range of particle sizes present in the injected sample aliquot may be separated in the same run. Indeed, for each class of particles to be separated an optimal separation may be developed by empirically varying the foregoing factors. The only variable that cannot be changed for a specific AF4 device is the channel length.

Historically, the channel length for A4F has been on the order of 25 to 30 cm with a greatest breadth of the order on 1 to 3 cm that tapers along its length and ends at a breadth comparable to the breadth of the exit port. Recent studies have suggested that a channel of shorter length would provide certain benefits and, on this basis.

AF4 has been used for analysis of exosomes in serum. Therefore, it is a useful method for rapid separation of different miRNA carriers based on their hydrodynamic diameters, enabling the screening of RNA distribution among various carriers. Comparing the distribution profiles obtained from healthy individuals and cancer patients may help to reveal which types of carriers are more relevant to cancer development, and thus enhance the sensitivity and specificity in diagnosis when using the miRNAs enclosed in those carriers as the markers.

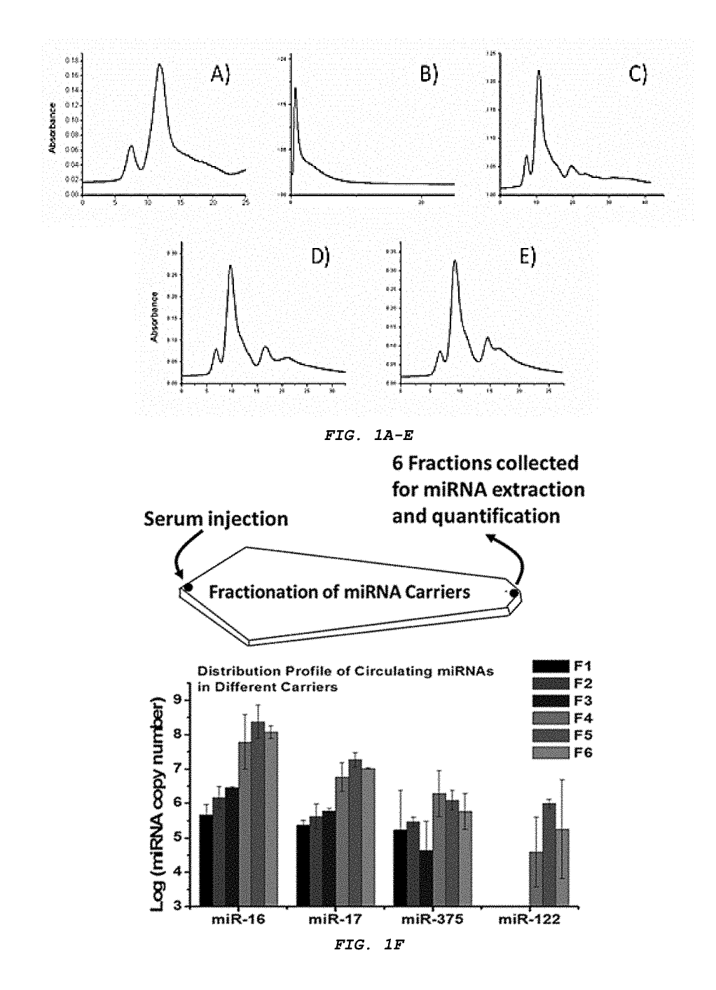

Accordingly, AF4 can be used for separation of different carriers in human serum. In one embodiment, AF4 is used to separate RNA carriers. RNA on (or in) such carriers can then be isolated. For example, the eluted RNAs are collected and quantified to obtain their distribution profiles among the various molecular carriers. FIG. 1F, depicts the information obtained for various miRNAs obtained from different fractions associated with different RNA carriers or sets of carriers obtained from A4F.

The disclosure also provides a device to carry out quick fractionation of RNAs based upon the main carriers. In comparison to the existing separation techniques used for miRNA fractionation, the methods and compositions of the disclosure are much faster and easier to perform; require smaller sample volumes and can be done with higher degree of automation to avoid variations introduced by human operators; and are more suitable for processing a high number of patient samples. The disclosure provides methods and devices for comprehensive screening of the distribution of circulating RNAs among various carriers. Such methods and devices facilitate the discovery of specific RNA biomarkers for disease diagnosis, and help to understand the biogenesis and functions of circulating RNAs, contributing to better diagnosis, therapy and prognosis.

Although the AF4-based method provides comprehensive distribution profiling by separating the carriers into various fractions, recovering RNAs from the large elution volumes is labor intensive and time consuming. Additionally, improved resolution between different carriers would provide better quantification. To further improve sample recovery, work efficiency, carrier resolution, and analysis throughput, while reducing sample consumption, a microchip-based distribution profiling technique was developed. This technique combines immuno-capture of the exosomes with detergent-based disruption of the protein-RNA binding to separately isolate the RNAs bound to proteins, associated with lipoprotein complexes, and enclosed in exosomes in three microchannels on a microchip. The total isolation process in the preliminary devices and methods took about 1.5 hrs with minimum manual sample handling; and only 25 μL or less serum is required. Improvements are being made in both the volume and time for processing. The eluted RNAs are of good quality and can be quantified by RT-real-time PCR or other RNA detection techniques.

The disclosure thus further describes a microfluidic/chip system is used to separate RNA carriers. In one embodiment a fluidic device is used that comprises at least one channel (e.g., 2, 3, 4, 5 or more channels), a sample reservoir for receiving a biological sample (e.g., serum) and at least one bead reservoir that comprises beads and/or can be used to remove and store beads that can bind to RNA carriers or RNA in the sample.

The microchip-based RNA distribution profiling method quantifies the circulating RNAs bound to three well-recognized carriers in a quick, high-throughput, and semi-automatic manner. The three channels on the chip separately yield the protein-bound, lipoprotein-associated, and exosomal RNAs, taking advantage of immuno-affinity and chemical reagents. As described more fully below, the on-chip method indeed yields the intended distribution profiling, and the obtained profiles can be used to distinguish between the serum samples collected from cancer patients and from healthy individuals.

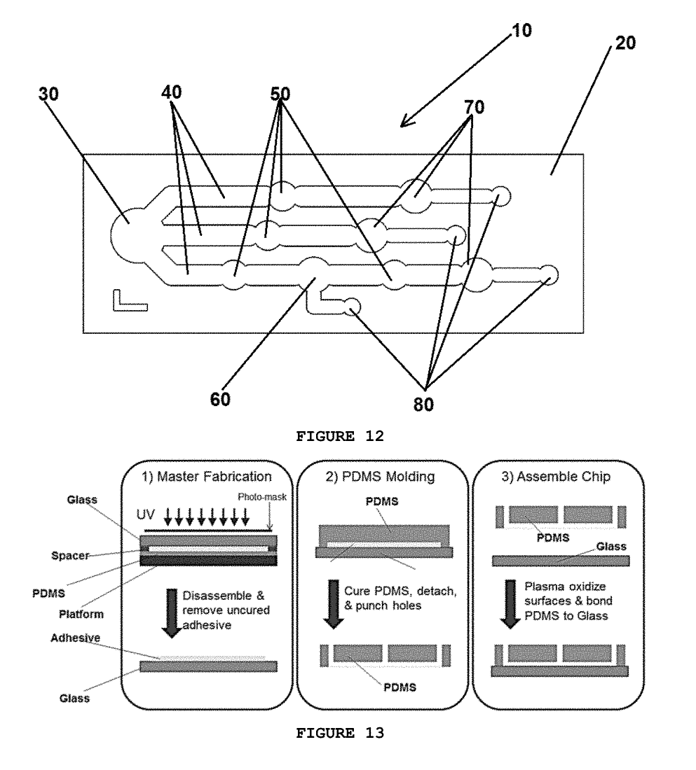

FIG. 12 shows an exemplary microfluidic device 10 of the disclosure. Channels 40 and various reservoirs 30, 50, 60, 70, 80 are formed on a substrate 20. Reservoirs 30, 50, 60, 70, 80 are fluidly connected by channels 40. For example, sample reservoir 30 is fluidly connected by channels 40 to one or more wash reservoirs 50. The device 10 includes one or more bead reservoirs 80 for holding magnetic or non-magnetic (e.g., silica) beads. The beads can be functionalized to include antibodies that bind to an antigen on the surface of a component in a sample or nucleic acids that hybridize to a desired target in the sample. The device can further include an exosome/vesicle disruption reservoir 60. This reservoir serves as a location for breaking apart vesicles containing RNA components. The device includes elution reservoirs 70, which serve to allow extraction of RNA from the sample for further processing by, for example, RT-PCR.

The channels 40 and reservoirs 30, 50, 60, 70, 80 contain different fluids/buffers. For example, channels 40 can comprise an oil (e.g., silicone oil, mineral oil etc.), while reservoirs 30, 50, 60, 70, 80 can comprise droplets formed from an aqueous-based buffer in the oil. In this way, different reaction components can be separated in the different reservoirs 30, 50, 60, 70, 80, while remaining fluidly connected by the channels 40.

The device 10 can be made using common microfluidic fabrication technology. FIG. 13 depict one embodiment of manufacturing the device 10. In this process of photo-mask and UV curing adhesive is used to define the regions of adhesion of a PDMS mold. The UV light cures only the adhesive in the desired areas and the uncured adhesive is remove (see, FIG. 13, step 1). A PDMS layer is then added and cured. The PDMS layer is removed and desired holes are punched in the PDMS (see, FIG. 13, step 2). The PDMS mold is then applied and bonded to a glass substrate (see, FIG. 13, step 3, also see, e.g., 20 in FIG. 12).

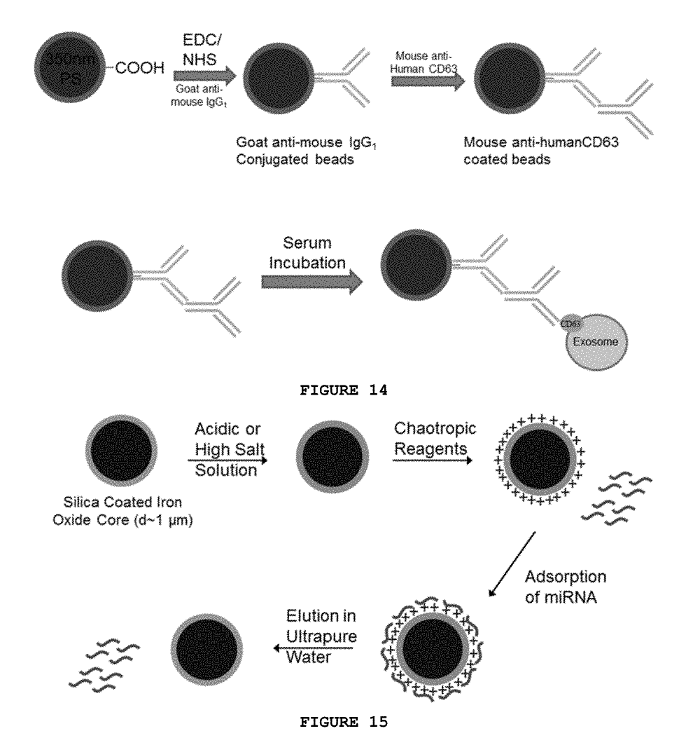

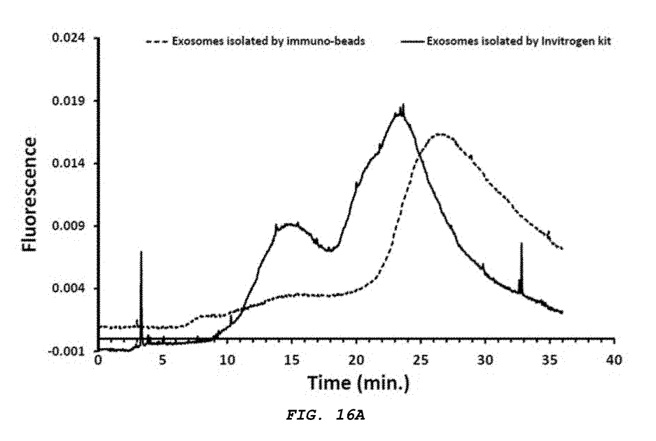

During operation a sample (e.g., serum) is provided into sample reservoir 30. In order to prevent unwanted and non-specific adsorption of either miRNA or other factors (e.g., serum components) on the surfaces of the channels or wells, the surfaces can be modified with an octamethyl siloxane species to block the surface and render the channels and wells hydrophobic and inert. Referring again to FIG. 12, the sample in the sample reservoir 30 is extracted into, e.g., 3 fractions (exosome, lipoprotein and protein). The exosome fraction pulled into the lower channel 40 using magnetic beads labeled with antibodies to an exosome surface antigen (see, FIG. 14). The exosomes tagged with immuno-beads are pulled through the channel 40 using a magnet to disruption reservoir 60 where the exosomes can be disrupted in ethanol and guanidine thiocyanate. The magnetic immuno-beads are then removed and magnetic silica beads with a cationic surface charge (see, FIG. 15) are contacted with the disrupted exosome contents. The cationic charged beads attract and bind the anionic charged RNA molecules present in the disruption reservoir 60. The beads can then pull the bound RNA molecules to extraction reservoir 70. A protein fraction of the sample is then obtained by contacting the sample with magnetic beads that (a) selectively bind to target proteins (using, e.g., anti-AGO2 antibodies) or (b) that can adsorb RNA via electrostatic attraction, H-bond, and/or hydrophobic interaction after the protein-RNA interaction is disrupted by the surfactant/chaotropic salt mixture. The beads are then pulled through channels 40 to elution reservoir 70. A lipoprotein fraction of the sample is then obtained by contacting the sample with magnetic beads that (a) selectively bind to an epitope on lipoproteins (using, e.g., anti-Oxidized phospholipid antibodies) or (b) that can bind to RNA via electrostatic attraction, H-bond, and/or hydrophobic interaction after the lipoprotein complexes are destroyed by the surfactant/organic solvent/chaotropic salt solution. The beads are then pulled through channels 40 to elution reservoir 70. Elution reservoir 70 contains a buffer (e.g., ultrapure water) that causes the release of the RNA from the beads. The RNA can then be isolated and RT-PCR'd and sequenced from each elution reservoir 70, thereby providing RNA sequence-carrier information.

For example, for serum protein bound RNA extraction, approximately 400 μg of 1 μm bare magnetic silica beads in about 0.6 MKCl, about 0.01% Tween 20, and about 4.5M Guanidine HCl are used. For serum lipoprotein bound RNA extraction approximately 400 μg of 1 μm bare magnetic silica beads, in about 1M KCl, about 0.11% Tween 20, about 3M Guanidine HCl, about 2.5M Guanidine Thiocyanate, and about 10% EtOH are used. For serum exosome, the captured exosomes are incubated in a solution comprising about 50% EtOH/3 M Guanidine Thiocyanate, remove capture beads and add about 400 μg magnetic silica beads, about 0.1% tween 20, and about 0.6M KCl.

Disclosed herein are methods to rapidly fractionate the microRNAs based on where they locate. The methods of the disclosure employ asymmetrical flow field flow fractionation to separate the microRNA carriers in serum. The eluted fractions can then be collected. For example, if a total of 6 fractions are collected, each fraction will comprise an enriched population of a particular carrier (e.g., faction #3 is enriched with high density lipoprotein (HDL) particle, and fraction #6 is enriched with exosomes). From the eluted fractions, the microRNAs from each fraction can be extracted and quantitated. In the experiments presented herein, it was further found that quantitated microRNAs from the fractions showed significant differences between healthy individuals and those with a disorder. But if the fractions were combined, the quantitated miRNAs in sum did not demonstrate as significant a difference between healthy subjects and those with a disorder.

In a particular embodiment, the methods disclosed herein utilize AF4 or a microfluidic process to fractionate the whole serum. By utilizing the methods of the disclosure, discrete elution fractions were collected; total RNAs were extracted from each fraction; and the amounts of 8 selected miRNAs in each fraction were quantified by RT-qPCR. Alternatively, the extracted RNA can undergo deep sequencing. Proteins eluted in each fraction were also extracted and identified to reveal the identities of carriers enriched in each fraction. Accurate quantification of the miRNA in each fraction yielded the distribution profile. The distribution profiles acquired from the sera of healthy individuals were compared with those from patients with breast cancer.

The term “deep sequencing,” as used herein, refers to nucleic acid sequencing to a depth that allows each base to be read hundreds of times, typically at least about 500 times, more typically at least about 1000 times, and even more typically at least about 1500 times. Deep sequencing methods provide for greater coverage (depth) in targeted sequencing approaches. “Deep sequencing,” “deep coverage,” or “depth” refers to having a high amount of coverage for every nucleotide being sequenced. The high coverage allows not only the detection of nucleotide changes, but also the degree of heterogeneity at every single base in a genetic sample. Moreover, deep sequencing is able to simultaneously detect small indels and large deletions, map exact breakpoints, calculate deletion heterogeneity, and monitor copy number changes. In some aspects, deep sequencing strategies, as provided by Myllykangas and Ji, Biotechnol Genet Eng Rev. 27:135-58 (2010), may be employed with the methods of the present disclosure.

It was found that by using the methods of the disclosure that the quantity of some miRNAs in particular fractions exhibited more distinct difference between healthy individuals and breast cancer patients, than the overall quantity, indicating that such miRNAs, when present in some type of carriers, could be more specific and sensitive biomarkers for cancer diagnosis. The knowledge of the carrier could help to improve the understanding on the fundamentals behind differential secretion of the miRNA markers by cancer cells and their transportation pathways in the circulation system. Such information can help to interpret their functions, and help with discovery of more effective therapeutic approaches. Accordingly, compared to current SEC based fractionation methods for collecting and quantifying miRNAs bound to carriers, the methods of the disclosure allows for a comprehensive screening of the miRNAs distributed in serum and the simultaneous evaluation of the quantity of different carriers. The methods of the disclosure therefore provide rich information that is not only useful for discovering biomarkers associated with disorders, such as indicating the particular cancer stage, but also for understanding the dynamics of the section and transportation of the circulating microRNAs.

To exemplify one embodiment of the disclosure a study of two groups of human samples, one from healthy individuals (control) and the other from cancer patients (case) have revealed that, different types of miRNA carriers, such as the lipoprotein particles and exosomes, could be enriched in individual eluted fractions after AF4 separation. The quantities of eight selected miRNAs in some of the fractions also showed larger changes between the “control” and the “case”, compared to the sum values. Moreover, statistical analysis on the distribution profiles revealed more potential miRNA markers than analysis on the overall miRNA quantity.

Sera from two healthy individuals (control) or from two cancer patients (case) were fractionated. Six fractions enriching different types of miRNA carriers, such as the lipoprotein particles and exosomes, were collected. The quantities of eight selected miRNAs in each fraction were obtained by RT-qPCR to yield their distribution profiles among the carriers. Larger changes in miRNA quantity between the control and the case were detected in the fractionated results compared to the sum values. Statistical analysis on the distribution profiles also proved that, the quantities of 4 miRNAs within particular fractions showed significant difference between the controls and the cases. On contrary, if the overall quantity of the miRNA was subject to the same statistical analysis, only 2 miRNAs exhibited significant difference. Moreover, principle component analysis revealed good separation between the controls and the cases with the fractionated miRNA amounts. Accordingly, the methods disclosed herein allow for the comprehensive screening of the distribution of circulating miRNAs in carriers. The obtained distribution profiles enlarge the miRNA expression difference between healthy individuals and cancer patients, facilitating the discovery of specific miRNA biomarkers for cancer diagnosis.

The following examples are intended to illustrate but not limit the disclosure. While they are typical of those that might be used, other procedures known to those skilled in the art may alternatively be used.

### EXAMPLES

### Example 1

Chemicals and Biomaterials.

HDL and low-density lipoprotein (LDL) were purchased from CalBioChem (EMD Millipore, Billerica, Mass.). Trizol LS reagent, 3,3′-dioctadecyloxacarbocyanine perchlorate (DiO) and Total Exosome Isolation kit were purchased from Invitrogen (Life Technologies). MicroRNA standards were purchased from Integrated DNA Technologies (Coralville, Iowa). TaqMan MicroRNA Assays specific to each miRNA strand were purchased from Applied Biosystems (Life Technologies). All chemicals used to prepare the AF4 running buffer of 1×PBS (10 mM phosphate at pH 7.4, 137 mM NaCl, 2.7 mM KCl, and 1.0 mM MgCl₂), ethylene glycol, dimethyl sulfoxide, guanidine hydrochloride, RNA-grade glycogen, 2-propanol, and chloroform were purchased from Thermo Fisher (Pittsburgh, Pa.). All single proteins used as AF4 standards were purchased from Sigma-Aldrich (St. Luis, Mo.). Taq 5× master mix was purchased from New England Biolabs.

Serum Samples.

The serum sample used for exosome extraction and separation method optimization was the pooled healthy male serum from Sigma-Aldrich. The serum samples used in the distribution profile study were from voluntarily consenting patients (females) under institutional review board-approved protocols. Both breast cancer patients had infiltrating ductal carcinoma and were ER/PR/HER2-positive (ER-estrogen receptor; PR-progesterone receptor; HER2-human epidermal growth factor receptor).

Serum Fractionation by AF4.

An AF2000 system manufactured by Postnova Analytics (Salt Lake City, Utah) was used in this study. The trapezoidal separation channel was 0.350 mm thick (thickness of the spacer), and its tip-to-tip length was 275 mm, with an inlet triangle width of 20 mm and an outlet width of 5 mm. The injection loop volume was 20 μL. The surface area of the accumulation wall was 3160 mm², which was made out of the regenerated cellulose ultrafiltration membrane (Postnova Analytics) with the molecular weight cutoff (MWCO) value of 10 kDa. The eluate exiting AF4 passed through a SPD-20A absorbance detector (Shimadzu) followed by a fraction collector (Bio-Rad). The running buffer for all samples was the 1×PBS mentioned above.

During serum fractionation, an initial focusing step of eight minutes was used, with the cross flow (the flow exiting the channel through the membrane wall) at 3.00 mL/min, tip flow (the flow entering the channel from the inlet) at 0.30 mL/min, and focus flow (a flow entering at a position further down from the inlet to focus the analyte into a narrow sample zone) at 3.00 mL/min. After focusing, there was a 1 minute transition period where the tip flow increased to 3.30 mL/min and the focus flow was reduced to zero. Afterwards, the tip flow was kept at 3.30 mL/min for five minutes, and was then reduced to 0.30 mL/min over the course of 15 minutes. In each case, the cross flow was reduced to keep the detector flow (the flow exiting the channel from the outlet) at 0.30 mL/min. A fraction collector (Bio-rad) was used to perform step-wise collection at every minute interval. These 1-min collections for each sample were then combined into 6 fractions, with fraction #1 (F1) containing the eluents collected from 6 to 9 min, F2 from 9 to 13 min, F3 from 13 to 16 min, F4 from 16 to 19 min, F5 from 19 to 23 min, and F6 from 23 to 28 min.

Protein and Particle Standards Used in Method Optimization.

Protein standards, as well as the pure HDL and LDL from Sigma, were prepared in solutions of 0.1 mg/mL for cytochrome C, albumin, transferrin, IgG, or thyroglobin. The 50-nm polystyrene beads were suspended at a concentration of 0.1 μM. Exosomes were prepared using an exosome precipitation kit (Invitrogen). In brief, whole serum was incubated with an exosome isolation reagent at a 5:1 v/v ratio for 20 minutes. The sample was then centrifuged at 4° C. to precipitate the exosomes. The supernatant was removed, and the exosomes were re-suspended in 1×PBS to give a 2× concentrated solution. The exosomes were either run in the system as-is or pre-incubated with DiO (final concentration of 5 μM) for 20 minutes at room temperature. All standards were analyzed using the same flow program but without the 5-min constant flow window.

LC-MS/MS Identification of Proteins.

Protein samples were subjected to tryptic digestion prior to LC-MS/MS analysis. Ammonium bicarbonate was added to reach a final concentration of ˜50 mM. Samples were reduced and alkylated using the standard DTT/IAA reduction alkylation protocols. Trypsin was added to the samples, and the digestion proceeded overnight at 37° C. After digestion, samples were purified using a C18 ZipTip (Millipore), and eluted in 50% acetonitrile/0.1% trifluoroacetic acid. After elution, samples were dried and resuspended in 0.1% TFA. These samples were then subjected to nano-LC-MS/MS analysis using a Waters 2695 Separations Module interfaced with a Finnegan LTQ (Thermo).

The raw data was uploaded to the Protein Prospector search engine (provided online by the University of California, San Francisco) for peptide and protein identification. Spectral counting was conducted for relative protein quantitation using the number of identified peptides for each protein (keeping replicates). In addition, specific searches were conducted for lower-abundance proteins of interest.

RNA and Protein Extraction from Collected Fractions.

Each fraction was spiked with 0.31 fmol C. elegans miRNA, cel-miR-67, and subjected to phenol-chloroform extraction using the Trizol® LS reagent (Invitrogen). Each fraction was split into several ˜450 μL aliquots, each aliquot homogenized with 1 mL Trizol LS reagent followed with the addition of 300 μL chloroform. After phase separation, the RNA-containing aqueous phase was mixed with RNA grade glycogen and the RNAs were precipitated by isopropanol (IPA). The RNA pellet was washed once by 80% ethanol, dried, and then all pellets for the same fraction were combined before going through another round of IPA precipitation and ethanol wash. The fractions were then dried and stored at −20° C. until RT-qPCR analysis. The protein-containing organic faction was precipitated using IPA and washed with 0.3 M guanidine hydrochloride in ethanol. After drying, the protein pellets were reconstituted in water.

MicroRNA Analysis.

To acquire sufficient miRNA amounts, two collections were carried out for each serum in each repeat. One collection was used to quantify hsa-miR-16, miR-191, let-7a, miR-17, miR-155, and miR-375, in which the miRNA pellets were reconstituted in 31 μL TE buffer. The other collection was for quantification of hsa-miR-21 and miR-122; and reconstitution of the miRNA pellets was done in 16 μL. The cel-miR-67 spiked into each fraction before RNA extraction was used as an internal standard to correct for sample loss during extraction, and the absolute miRNA quantity in each sample was obtained using an external standard calibration curve prepared from reactions with standard miRNAs.

The six high-abundance strands (hsa-miR-16, miR-191, let-7a, miR-17, miR-155, and miR-375) were all analyzed from a single collection. The remaining two strands (hsa-miR-21, and miR-122) were analyzed from another single collection. Prior to reverse transcription, lyophilized miRNA pellets were reconstituted in either 31 μL (for the high abundance strands) or 16 uL (for the low abundance strands). In each RT reaction, 5 μL of sample was mixed with 3 μL of a reverse transcription master mix and 2 μL of a corresponding RT primer for reach miRNA strand (TaqMan reverse transcription probe). The master mix consisted of 1.1 μL nuclease-free water, 1 μL of a 10× buffer mix, 0.13 μL of RNAse inhibitor, 0.1 μL of a dNTP mix, and 0.67 μL reverse transcriptase (all components were provided in a TaqMan reverse transcription kit). After mixing, 5 μL of silicone oil was layered on top of the RT mixture, and reverse transcription conducted on a Perkin-Elmer 2400 GeneAmp PCR system. The RT reaction consisted of a 30-minute annealing step at 16° C., a 32-minute transcription step at 42° C., and a 5-minute denaturing step at 85° C.

After RT, the samples underwent quantitative PCR (qPCR). On the qPCR plate, 1 μL of the RT product was mixed with 9 μL of qPCR master mix for a final volume of 10 μL. As an overlay, 5 μL of silicone oil was added to the top of each sample to limit evaporative loss. The master mix consisted of 4.9 μL of nuclease-free water, 1 μL of ethylene glycol, 0.1 μL of DMSO, 0.5 μL of 25 mM magnesium chloride, 2 μL of Taq 5× master mix, and 0.5 μL of TaqMan microRNA Assay 20×qPCR reagent (containing miRNA RT product specific forward and reverse PCR primers, and also a RT product specific TaqMan fluorescent probe). Each sample was plated in triplicate, as were any standards corresponding to the samples analyzed (high-versus low-abundance). The qPCR analysis was conducted on a Bio-Rad CFX real-time instrument, with an initial activation step at 95° C. for 90 seconds followed by a initial annealing step at 59° C. for 50s, then followed by a 40-cycle PCR with 30 second denaturation at 95° C. and 70 second annealing/extension at 53° C. for each cycle. Cel-miR-67 was used as an exogenous standard to account for sample loss during extraction, and miRNA levels were normalized and quantified using a standard calibration curve.

ELISA for Exosome Detection.

The total amount of proteins in each of the 6 collected fractions added into the well of the microtiter plate (Thermo, Microfluor 2 coated, flat bottom) were around 22 ng and diluted up to 50 μL with 1×PBS. The ELISA plate was incubated overnight at 4° C. to let the proteins be adsorbed onto the bottom of the well. Then, the protein solution was discarded, and the plate was washed with 200 μL 1×PBS for two times (all washing buffers used in the assay were 1×PBS), before 200 μL of the blocking buffer containing 5% non-fat milk in 1×PBS was added for each well. After 2-hr incubation at room temperature with gentle shaking, the blocking buffer was dumped and the wells were washed twice. Next, 100 μL of the primary antibody (mouse anti-human CD63, Catalog #ab8219, Abcam, Cambridge, Mass.) in 1:5000 dilution with 1×PBS was added to the wells, followed with another 2-hr incubation at room temperature. Following 4 washes, 100 μL of the secondary antibody, HRP conjugated rabbit anti mouse IgG (Catalog # ab97046, Abcam) in 1:25000 dilution was added and incubated for 1 hour at room temperature with gentle shaking. The plate was washed 4 times before 30 μL of the Perice ECL substrate (Thermo Fisher) was added, and incubated for 5 minutes. The resulted chemiluminescence was detected. Two repeats were done on the same plate. For the standard curve, two repeats of human CD63 (Sino Biology) with gradient concentrations were added in the same plate. The blank contained only 1×PBS in the adsorption step.

AF4 Separation of miRDA Carriers.

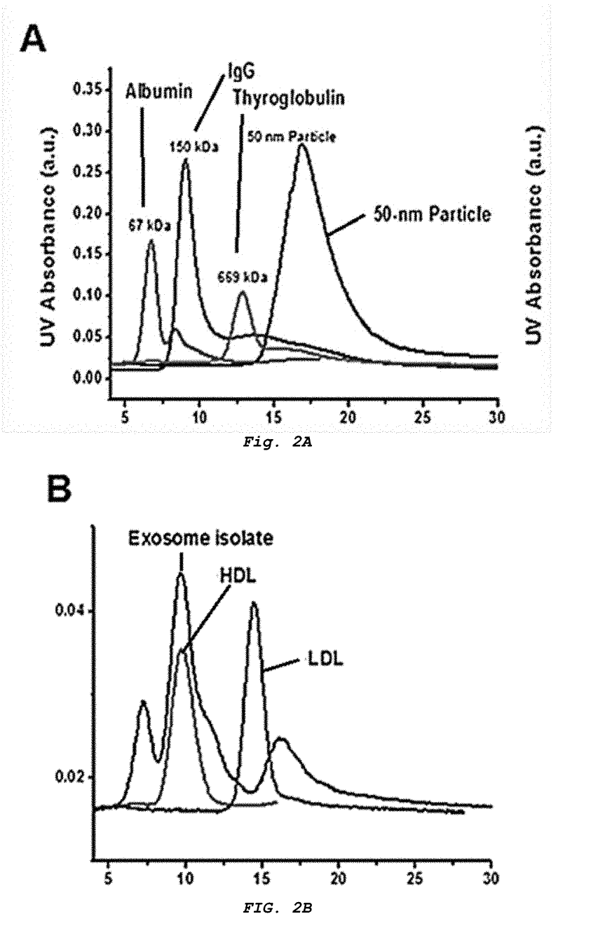

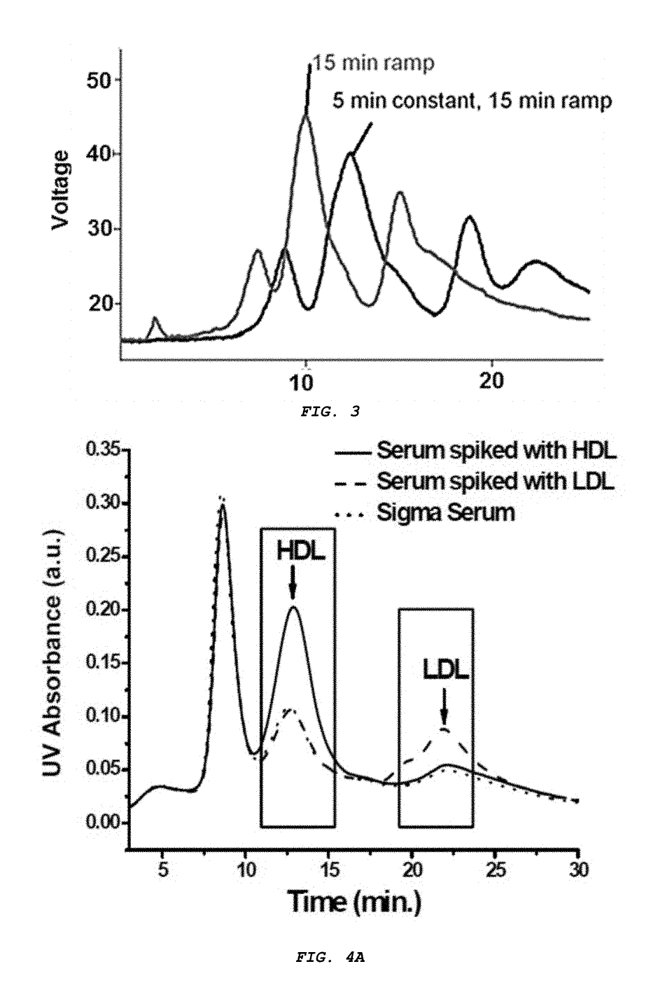

Due to the large differences in the hydrodynamic diameter (dh) between proteins and exosomes, the AF4 separation condition needs to be optimized to elute all carriers in a reasonable period of time while maintaining modest resolution between different species. In particular, elution of large particles like exosomes could take a very long time, since their diffusion rate is slow. Under a constant channel/cross flow condition, the exosomes prepared by the Total Exosome Isolation kit was injected but not eluted within 30 minutes, unless the cross flow was turned off gradually (See FIG. 1A-B). It turned out that better resolution between exosomes and the smaller serum components, as well as quick elution of the exosomes with limiting peak tailing, was achieved if the cross flow gradually decreased to zero within 15 minutes (see FIG. 1C). Using this flow program, protein standards with various dh: albumin (Mw 67 kDa, dh˜4 nm), IgG (Mw 150 kDa, dh˜8 nm) and thyroglobulin (Mw 660 kDa, dh˜16 nm), as well as the polystyrene nanoparticle (dh=50±7 nm, representing the average exosome diameter), were eluted at different times (see FIG. 2A); so did the HDL (dh˜7-10 nm), LDL (dh˜21-28 nm), and exosomes (FIG. 2B). The results support that the major serum carriers could be eluted in the order of single proteins<HDL<LDL<exosomes (ranking by elution time). To further improve separation resolution between the larger lipoprotein particles and exosomes, a 5-min constant flow period, i.e. the cross flow was maintained at 3.0 mL/min for 5 min before starting to ramp down (see FIG. 3) was used. This condition also gave out slight improvement when resolving the single proteins and HDL. This new method was then used in the subsequent experiments.

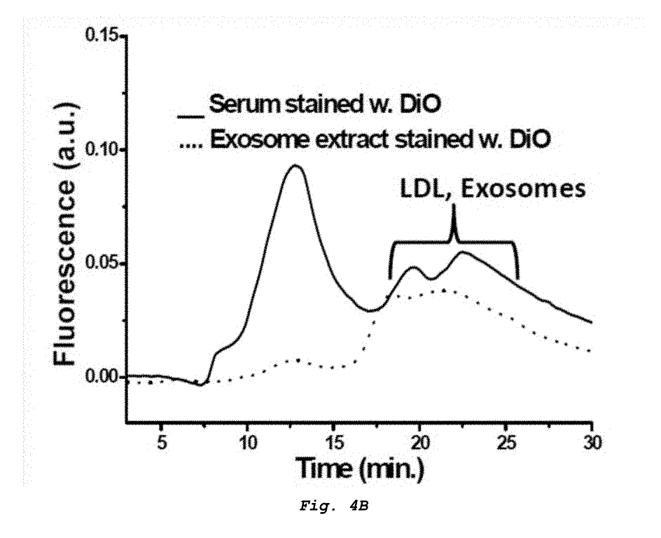

The whole human serum purchased from Sigma was fractionated by the optimized AF4 method. The serum was spiked with pure HDL and LDL to determine their exact elution windows (see FIG. 4A). HDL was eluted within 10-15 min and LDL between 17 and 23 min. Moreover, the serum or the exosome extracts were stained with the lipophilic dye of DiO prior to AF4 fractionation. DiO is weakly fluorescent in water, but emits strong fluorescence with high photo-stability when incorporated into lipid membranes. The fractograms obtained with fluorescence detection (λex=490 nm; λem at 510 nm) further confirmed that, structures with lipid membranes were mainly eluted after 17 minutes (see FIG. 4B).

Fractionation of Patient Serum and Confirmation of Carriers Eluted in Each Fraction.

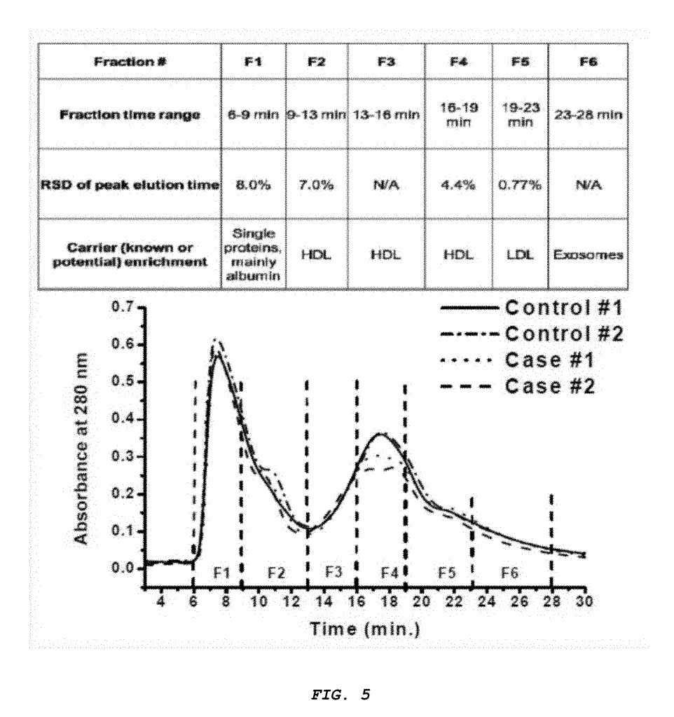

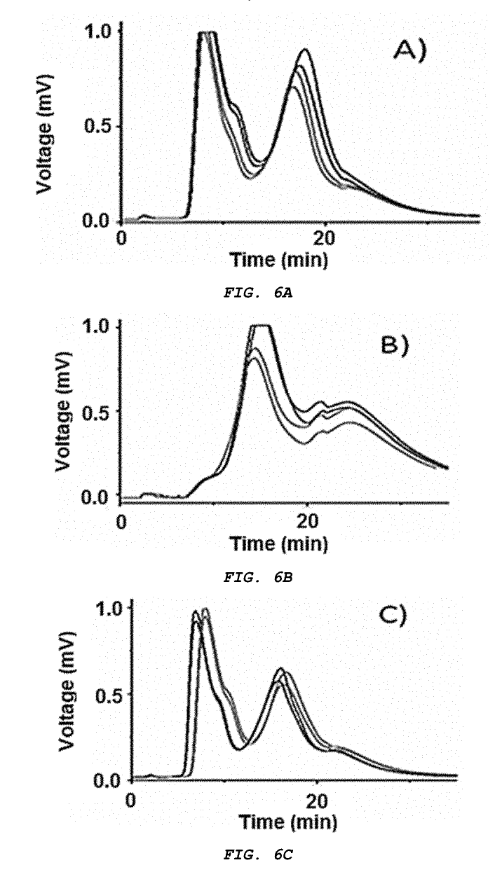

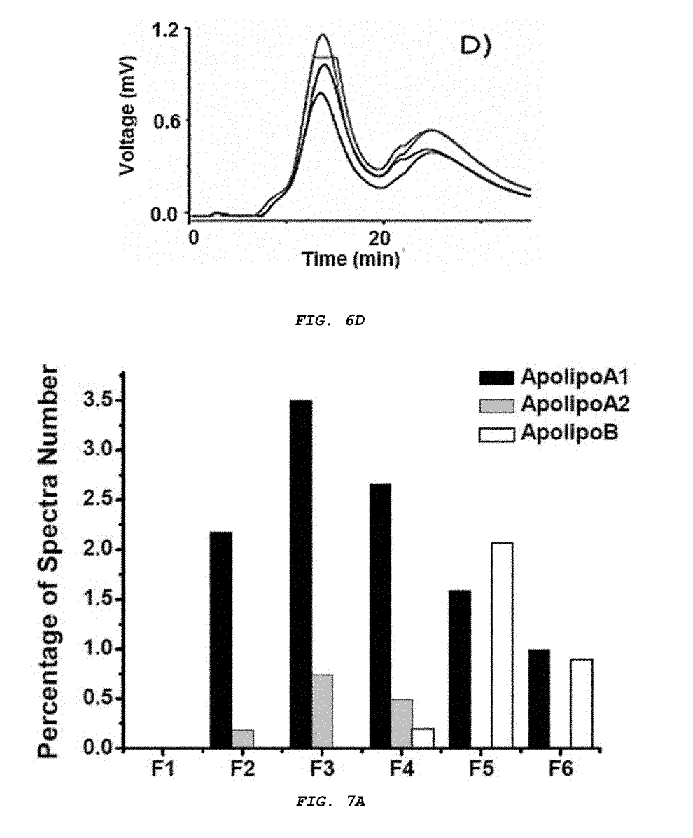

Once the approximate windows for elution of the known miRNA carriers were known, sera samples collected from 2 healthy females (control, referred as Control #1 and #2) and 2 breast cancer (BC) patients (case, Case #1 and #2) (see FIG. 5) were fractionated. Six fractions were collected to increase the purity of miRNA carriers enriched in each fraction. The collection window for each fraction was determined by the relative elution times of HDL, LDL, and exosomes obtained from the above study (inset table in FIG. 5). Separation was highly reproducible: relative standard deviation (RSD) of the elution time of the peak within each fraction was <8% using all 8 fractograms collected (four serum samples, each with two repeats) (see FIGS. 6A and 6C). DiO staining was performed for all the 4 serum samples tested, and confirmed the reproducible elution of the carriers with rich lipid structures, such as HDL, LDL, and exosomes (see FIGS. 6B and 6D). The highly reproducible separation profiles obtained by both UV absorption and DiO staining coupled with fluorescence detection helped to confirm the similarity in regular protein (represented by the peak intensity of serum albumin and IgG) and lipid (represented by the two major peaks detected by DiO staining) contents among these samples. This can ensure that the difference detected in miRNA distribution profiles was originated from the presence of BC but not from difference in carrier abundance. Moreover, the high reproducibility greatly simplified the after-column collection: a fraction collector was programmed to automatically collect the eluent every one minute, and the fractions within the desired time windows were combined for subsequent miRNA and protein extraction.

To confirm the identities of carriers enriched in each fraction, proteins eluted in F1-F6 were collected, digested by trypsin, and analyzed by LC-MS/MS. The relative abundance of the eluted proteins were evaluated by spectral counting, which counts the number of mass spectra collected for a specific protein. The percentage of the spectra number for a particular protein among all spectra identified in one sample should be semi-quantitatively proportional to its relative abundance in the mixture.

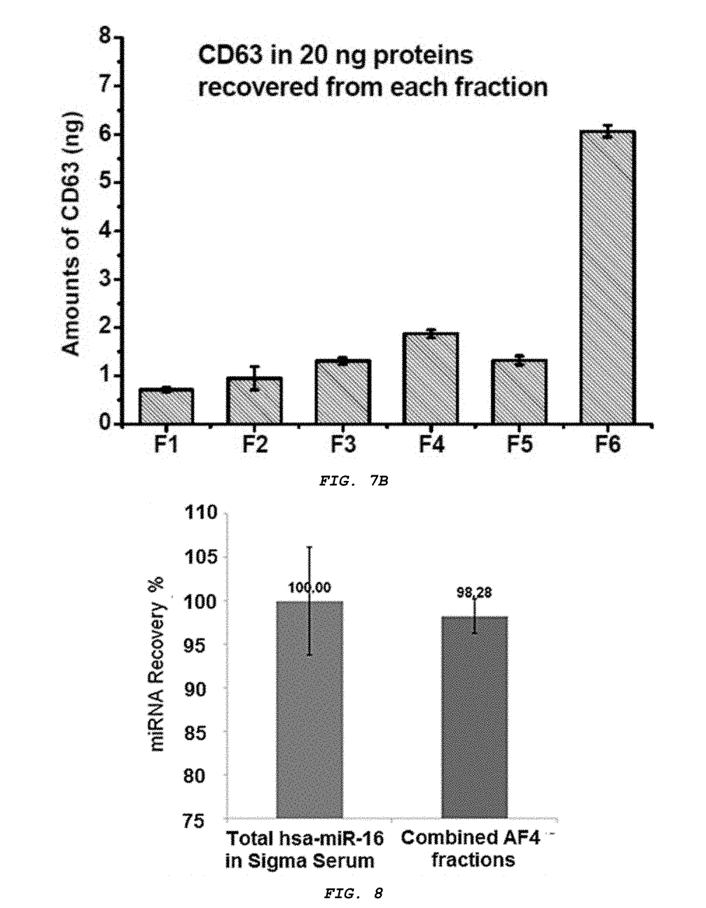

Apolipoproteins belonging to various lipoprotein complexes, such as apolipoprotein A-I (ApoA-I), A-II (ApoA-II) and B-100 (ApoB), were found in multiple fractions (see FIG. 7A). ApoA-I, as the marker for HDL, was found in F2-F6, probably because of its association with all lipoprotein complexes and even in exosomes. The other marker protein for HDL, ApoA-II, was present in F2-F4 fractions, and also more enriched in F3. Considering the size range of HDL reported in literature, i.e. 7-10 nm, it was concluded the heterogeneous high-density lipoprotein (HDL) particles were eluted in F2, F3, and F4. ApoB is the marker protein for LDL as well as the very-low-density lipoproteins (VLDL), and was found in F4-F6, with the majority eluted in F5. Thus, LDL should be enriched in F5, matching with migration window of the pure LDL shown in FIG. 2B and FIG. 4A.

LC-MS/MS did not identify marker proteins for exosomes, probably due to the signal suppression resulting from the highly abundant serum proteins like IgG and albumin. Instead, the marker protein for exosomes, CD-63, was detected in each fraction by ELISA (see FIG. 7B). About 20 ng of the protein extracted from each fraction (determined through the bicinchoninic acid assay) was adsorbed to the bottom of the microtiter plate well. CD-63 was detected by the anti-CD63 antibody and the HRP-labeled secondary antibody. A substantial amount of CD63 (˜6 ng/20 ng) was detected in F6. As was concluded from the standards analysis, F6 was where exosomes were primarily located.

Overall, the above results point out that, F1 contained mainly albumin or proteins with MW<100 kDa. HDL and LDL should be enriched in F3 and F5, respectively; and exosomes mainly in F6, but could also be in F5. VLDL was co-eluted with exosomes in F6. Although co-elution of multiple carriers was seen using the current separation method, such as the overlap of HDL and LDL in F4, and the co-elution of exosomes and VLDL in F6, enriching specific carriers in particular fractions should already allow the look at the general distribution of miRNAs among the carriers. Higher resolution will indeed enhance the accuracy in distribution profiling, and can be achieved by injecting lower amounts serum in each round of the separation, but multiple collections are needed, increasing the overall labor in the analysis, which is not a favorable choice. Increasing the separation force by using a higher crossflow may also be beneficial to separation resolution, but the risk of losing more miRNAs due to membrane adsorption is increased. Thus, the current fractionation conditions were used in the subsequent experiments. The results demonstrate that the coarse distribution profiles were adequate in differentiating the cancer patients from healthy controls, as well as in revealing strands and particular carriers that were important to the differentiation.

Distribution of miRNAs in Serum.

The total RNAs were precipitated and reconstituted in water for quantification by RT-PCR. As stated above, sera from two groups of donors (all females) were tested. The sera from healthy individuals (Control #1 and #2); and those from breast cancer patients (Case #1 and #2) were analyzed, each with two repeated measurements. Eight miRNAs were quantified by RT-qPCR. Their sequences are listed in TABLE 1, together with the rationale of their inclusion in the study.

TABLE 1
MicroRNA strand information

| Strand | Sequence | Rationale for study |
|---|---|---|
| cel- | 5′-cgcucauucugc | As internal standard |
| miR-67 | cgguuguuaug-3′ | for correction of |
| | (SEQ ID NO: 1) | extraction efficiency |
| hsa- | 5′-ugagguaguagg | Reported in BC markers, |
| let-7a | uuguauaguu-3′ | unregulated in |
| | (SEQ ID NO: 2) | references shown in |
| | | Table 1 and in |
| | | miRCancer; an exosomal |
| | | miRNA in Arroyo, 2011 |
| hsa- | 5′-uagcagcacgua | Reported in miRNAdola |
| miR-16 | aauauuggcg-3′ | as a circulating miRNA; |
| | (SEQ ID NO: 3) | in miRCancer as a |
| | | potential BC marker; |
| | | in Arroyo, 2011 as |
| | | protein-bound miRNA |
| hsa- | 5′-caacggaauccc | Reported in miRNAdola |
| miR-191 | aaaagcagcug-3′ | as a circulating, |
| | (SEQ ID NO: 4) | exosomal miRNA; in |
| | | Elyakin, 2010 as a |
| | | potential BC marker |
| hsa- | 5′-caaagugcuuac | Reported in miRNAdola |
| miR-17 | agugcagguag-3′ | as circulating in BC; |
| | (SEQ ID NO: 5) | in Vickers, 2011 as a |
| | | HDL-bound miRNA |
| hsa- | 5′-uuaaugcuaauc | Reported in miRNAdola |
| miR-155 | gugauaggggu-3′ | as an exosomal miRNA, |
| | (SEQ ID NO: 6) | and as a potential BC |
| | | marker |
| hsa- | 5′-uuuguucguucg | Reported by Wang et |
| miR-375 | gcucgcguga-3′ | al. as a potential |
| | (SEQ ID NO: 7) | marker for prediction |
| | | of clinical outcome |
| | | of BC patients; in |
| | | miRNAdola as an |
| | | exosomal miRNA; in |
| | | Vicker 2011 as HDL- |
| | | bound miRNA |
| hsa- | 5′-uagcuuaucaga | Reported as potential |
| miR-21 | cugauguuga-3′ | BC markers that is |
| | (SEQ ID NO: 8) | upregulated; in |
| | | miRNAdola as a |
| | | circulating miRNA; in |
| | | Arroyo, 2011 as |
| | | protein-bound miRNA |
| hsa- | 5′-uggagugugaca | A potential BC marker |
| miR-122 | augguguuug-3′ | located mainly in |
| | (SEQ ID NO: 9) | exosomes |

Recovery of miRNAs in the method was evaluated by quantification of miR-16 in the serum from Sigma. The total content of miR-16 was directly extracted from the whole 20-μL serum by the TRIzol reagent was compared with the sum miRNA quantity recovered from all AF4 fractions obtained with the injection of the same serum volume. A recovery as high as 98% was achieved (see FIG. 8), indicating no significant loss of miRNAs due to membrane adsorption inside the AF4 channel. The resulted copy number of each miRNA tested in 20-μL serum normally ranged from 10⁴ to 10¹⁰. miR-375 and -122 were present at much lower abundances than other strands or even not detected in some of the fractions.

The high reproducibility in the separation step and careful processing in miRNA extraction and quantification ensured high analytical reproducibility: the RSD for the Log value of the total miRNA content in the two repeated measurements was <5% for most of the strands, except for miR-375, -21, and -122, which could vary by up to 15%. The results agreed with previous reports, large variations in the miRNA amounts were observed among individuals, even between the two samples within the same health group: the controls or the BC cases. Evaluation of the RSD of the total miRNA amount in all serum samples points out that, miR-16 and -17 had relatively more stable expression among individuals than other miRNA species. Their RSD was below 15%. However, this RSD already corresponds to about 10-fold alteration in the miRNA copy numbers if the base value is around 10⁶. For miR-122, RSD values close to 120% were observed between the two samples within the same group.

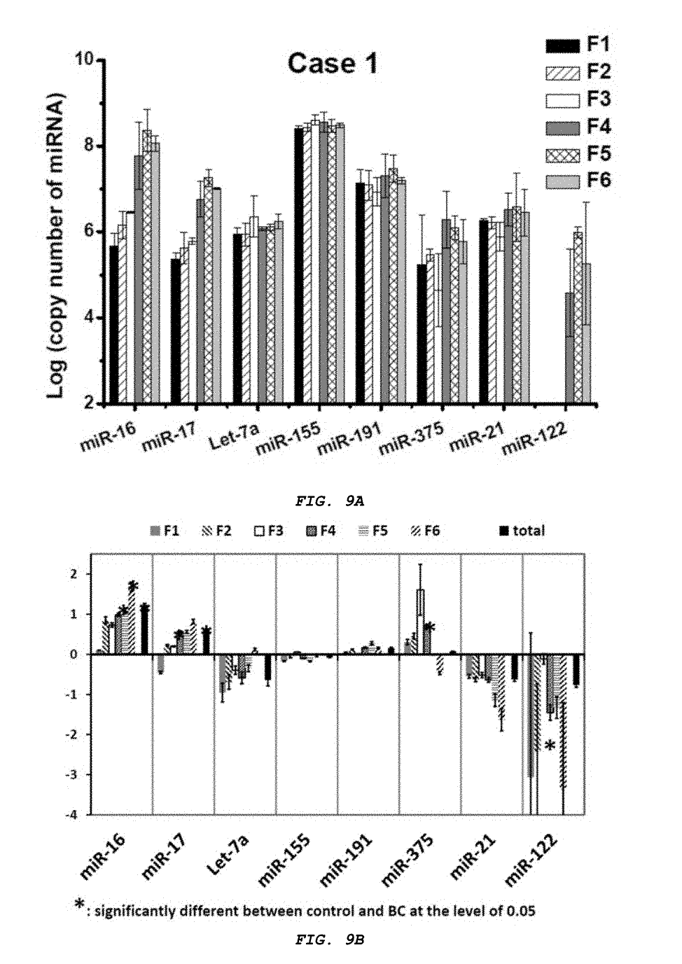

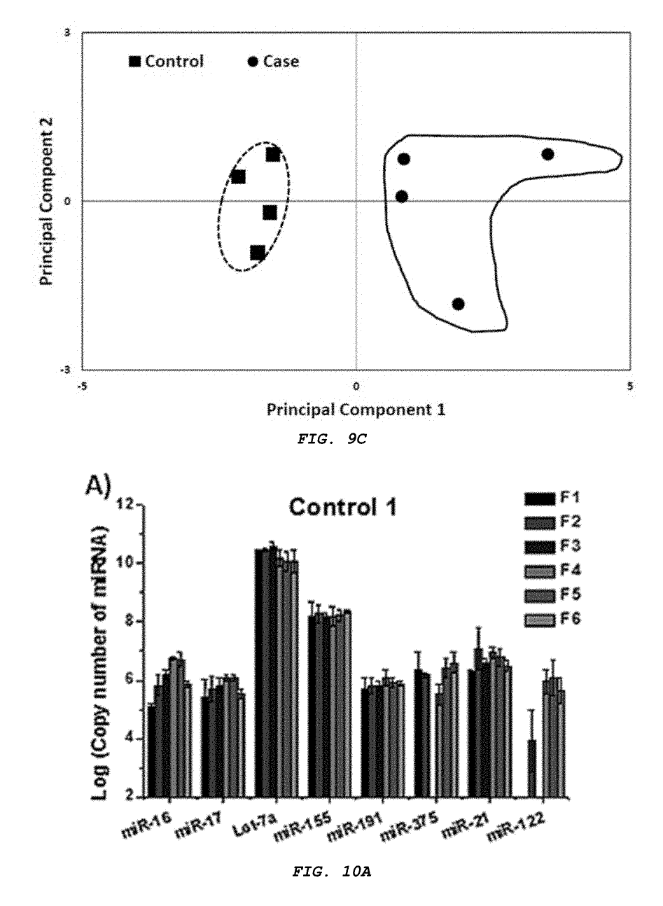

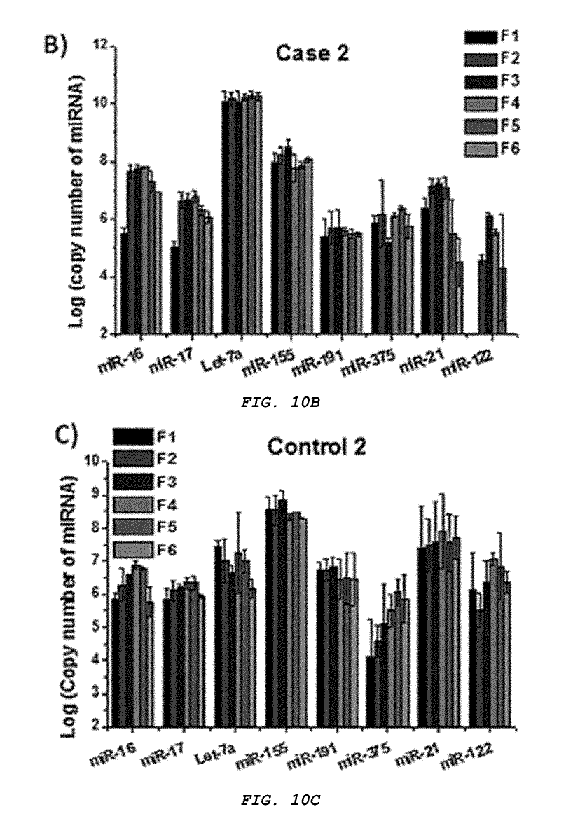

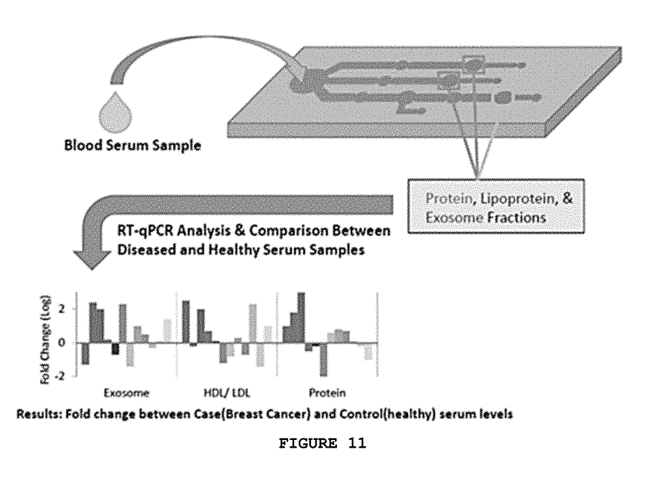

Since each fraction enriched a particular type of miRNA carrier, the copy number found in each fraction corresponded to the miRNA level in that particular carrier. Different miRNAs showed distinct distribution patterns among the carriers, as demonstrated by the distribution profile of Case #1 (see FIG. 9A; the profiles of other samples are shown in FIG. 10). In this sample, higher amounts of miR-16, -17, and -122 were found in F4-F6. There was even no detectible miR-122 in F1-F3. Thus, these three miRNAs should mainly locate in lipoprotein complexes and exosomes in this serum sample. By contrast, Let-7a, miR-155, and miR-191 had quite flat distribution among all fractions. The main type of carriers for each miRNA could be related to the major pathway it takes when exiting the cells, and be possibly linked to their biological functions. By fractionating the carriers prior to miRNA quantification, the method of the disclosure provides rich information about how the miRNAs are present in serum, which can be further explored to solve the fundamentals of miRNA secretion and transportation.

The miRNA copy number found in each fraction was then compared between the control and BC samples. FIG. 9B shows the Log ratio of the averaged miRNA copy number in the BC samples over that in the control samples; i.e. Log (BC/control), for each miRNA. If the miRNA level was lower in the BC cases than in the controls, a negative Log(ratio) value would be obtained, and vice versa. Larger absolute values of Log (Case/Control) indicate more obvious difference between these two groups. The Log(Case/Control) obtained using the total miRNA quantity from all fractions (displayed as red bars) was also included. The sum represents the result attainable with the standard approaches in miRNA study, in which the overall expression level of each miRNA is quantified. FIG. 9B clearly shows that, larger differences between the BC and control samples were observed in some fractions than in the sum value for all miRNAs tested, except for miR-155 and -191. These results hint that the miRNA quantity change in some of the carriers could be more sensitive in differentiating the cancer patients from healthy controls than the overall quantity in the whole serum. This speculation was supported by the following statistical analysis.

Statistical Analysis of the miRNA Distribution Profiles.

To see whether the distribution profile could tell the difference between healthy donors and BC patients, and whether more reliable miRNA biomarkers can be found, for the 8 miRNAs listed in TABLE 1, their quantities in each fraction were fitted in the linear mixed effects model of EQ. 1, using R 3.0.2.

Yᵢⱼₖ=μ+bᵢ+bⱼ₍ᵢ₎+εᵢⱼₖ (EQ. 1)

where i=1,2(# of patient group),
j=1,2(sample # in each group),
k=1,2 (replication),
bᵢ: effect of ith group (fixed, 1 for the control group, 2 for the BC case group),
bⱼ₍ᵢ₎: effect of jth sample in group i (random, 1(1) for Control #1,2(1) for Control #2,1(2) for Case#1, 2(2) for Case #2) bⱼ₍ᵢ₎˜N(0,σ_{b}²), εᵢⱼₖₗ˜N(0,σ²), bⱼ₍ᵢ₎ and εijk are independent.

For a miRNA in a given fraction, Y is the log value of the observed miRNA copy number. For example, for miR-16 in F1, Y111 is the Log value of the miRNA copy number from one of the two repeats of Control #1. This linear mixed effects model accounted for sample to sample variation σ_{b}², as well as within sample variation σ², when comparing healthy donors to BC patients, i.e., testing the hypothesis H₀:b₁=b₂=0. This hypothesis was tested for each fraction of each one of the eight miRNAs using likelihood ratio test. To compare with standard approach, the same test was also performed on the sum of all fractions for each miRNAs. More miRNA strands (miR-16, -17, -375, and -122) in particular fractions (miR-16 in F5 and F6, -17 in F4, -375 in F4, and -122 in F4) yielded significant difference between healthy donors and BC patients at the level of 0.05, as marked by the “*” sign in see FIG. 9B; while only miR-16 and 17 showed significant difference if the sum value was used.

It is interesting to see that miRNA quantity in F4 or F6 seems to matter the most in differentiating cases from controls. While F6 mainly contained exosomes, F4 enriched HDL and LDL. Then it is possible that, while all four markers may be valuable in diagnosis of breast cancer, they may be released by cancer cells via different pathways. miR-16 could be secreted in exosomes; but miR-17, -375, and -122 in the lipoprotein complexes could be more relevant to the development breast cancer than the exosomal fraction. This highlights the necessity of testing the miRNA quantities in multiple carriers, instead of in only one.

To visualize the effectiveness of the quantity of miR-16 in F5 and F6; miR-17 in F4; miR-375 in F4, and miR-122 in F4, in discriminating healthy donors and BC patients, principal component analysis (PCA) was performed using XLSTAT 2014 (Addinsoft™). The contents of each miRNA in individual fractions were considered as the variables. For example, the miR-16 content in F6 is one variable and named as miR-16-F6. A total of 8 observations were made in the study, two repeats for each sample were counted as two independent observations. PCA suggests that the first principle component with loadings −0.436, −0.598, −0.167, −0.258, 0.599 on miR-16-F5, miR-16-F6, 17-F4, 375-F4, and 122-F4, respectively, can potentially separate healthy donors from BC patients, as shown in the scores plot in FIG. 9C. In fact, the first principle component already accounts for 87.1% total variation. Analysis of a sample set containing a much larger number of both healthy controls and cancer patients, is then used to draw affirmative conclusions about the capability of these potential markers in cancer diagnosis.

### Example 2

Microchip Fabrication.

In brief, the microchip was fabricated as generally depicted in FIG. 13. The microchip platform was made by bonding a 3″×1″ glass slide (0.5 mm-thick) and a cured PDMS substrate together. In order for the PDMS substrate to be made, a total of three masters were prepared. Firstly, the first master, Master-1, that contained only the channel features, was fabricated from the thiolene-based optical adhesive, NOA81, by an open-faced method. In this method, NOA81 was pre-cured between a glass slide (plasma treated) and a PDMS working stage by 5-second radiation with a collimated UV light source (365 nm, ˜8.3 mW/The thickness of Master-1 was determined by spacers (˜400 μm) placed between the glass slide and the PDMS stage, and the features were defined by a photomask. After the short UV exposure, the glass slide was slowly removed from the PDMS stage, with the pre-cured NOA81-based channel features on the surface. The unexposed adhesive was removed by sequential rinsing with ethanol, ethanol/acetone mixture (1:1), and ethanol again, using a syringe. The air-dried glass slide was illuminated for 345 sec by UV exposure, a post-cure step aiming to increase the adhesion of NOA81 to glass. A subsequent 12-hr thermal cure at 50° C. was carried out to extend the structure's lifetime. Thereafter, Master-1 was treated by 1,7-dichloro-octomethyltetrasiloxane to produce a non-stick surface on the master mold of the device and utilized to mold the PDMS substrate for making Master-2. The PDMS substrate was cured at 60° C. for 4 hours and then peeled off the Master-1; holes were punched in the location of wells to form Master-2. After attaching Master-2 on a plasma-treated glass slide, NOA81 was injected into the channels and wells without trapping any bubbles, cured under UV for 1300 sec, and thermally aged at 50° C. for 12 hours. By carefully removing PDMS Master-2, Master-3 was accomplished with both the low channel features and the tall pillar structures on the surface, and can then be used for replication of the PDMS substrate for the microchips used for miRNA extraction. The chips were finally obtained by covalent bonding of the formed PDMS substrate on a thin glass slide through plasma oxidation. The channels of the device are methylated by washing and incubating with 1M NaOH for 10 minutes, washing with water, ethanol, and then drying, followed by incubation of the 1,7-dichloro-octomethyltetrasiloxane reagent (10% v/v in ethanol). The chip is then rinsed with ethanol and dried.

Preparation of Microbeads.

The polystyrene magnetic microbeads with an average diameter of 350 nm were conjugated to goat anti-Mouse IgG using carbodiimide crosslinking (see, e.g., FIG. 14). A mixture of 10 mg 1-Ethyl-3-(3-dimethylaminopropyl)carbodiimide (EDC) and N-hydroxysuccinimide (NHS) was added to 1 mg (20 mg/mL) microbeads suspended in 50 mM MES buffer (pH ˜5.5). After 30-min incubation, the activation buffer was removed and the beads were washed 2 times with the coupling buffer (lx PBS, pH ˜7.2). Then the appropriate amount of anti-mouse IgG was added to the beads resuspended in the coupling buffer at a final concentration of 10 mg/ml. The mixture was incubated with mixing for overnight at 4° C. The beads were then washed twice with the coupling buffer and then resuspended in 25 mM Glycine buffer (pH ˜7.2), and incubated for 30 minutes at RT. After two washes with 1×PBS containing 1% BSA, the beads were dispersed in storage buffer (1×PBS with 0.01% BSA) to the desired concentration of 25 mg/ml. Before usage, the microbeads would be mixed with the mouse anti-human CD63 IgG at a final concentration of 10 mg/ml in 1×PBS, followed with overnight incubation with mixing at 4° C. After binding, the beads were then washed 3× with 1×PBS and stored in PBS storage buffer.

Extraction of miRNAs.

The chip layout is shown in FIG. 12. Three channels for extraction are depicted each linking several wells/reservoirs, and filled with silicone oil. The wells were preloaded with the appropriate aqueous solutions in the form of droplets. Channel 1 and 2 had the wash, elution, and bead collection reservoirs, and were used for isolation of the protein- and lipoprotein-bound miRNAs. Channel 3 was for isolation of exosomal miRNAs. Channel 3 included the regular wash, elution, and bead collection reservoirs as the other two channels, but further included two more wells; one for exosome purification and disruption and PS-bead loading.

The extraction started by adding 25 μL serum and 100 μg immune-beads conjugated with the anti-human CD63 IgG to the sample reservoir. The sample was pipetted up and down for 3-5 times to mix well and incubated for 30 minutes at room temperature. The beads were then moved towards Channel 3, through a wash reservoir, and then into a disruption reservoir, using a permanent magnet underneath the microfluidic chip. The wash reservoir contained 1×PBS and the disruption reservoir held 30 μL solution consisting of 75% EtOH and 2 M guanidine thiocyanate and 1% tween-20. After 15-minute incubation in the disruption reservoir the beads were removed into the connected bead reservoir, and then 20 μL of 9 M GuHCl and 4 μL of 6M KCl were added to the well, followed by 200 μg of the 1 μm magnetic silica beads. After mixing and another round of 15-minute incubation, the silica beads travelled to the elution reservoir that contained RNase-free ultrapure water, mixed, and incubated for 15 minutes to unload the miRNAs, before the silica beads were removed into the corresponding bead reservoir.

Extraction of the protein and lipoprotein bound miRNAs was started while the exosomal miRNAs were being isolated. After removal of the exosomes from the serum, 30 μL of 9M GuHCl, 6 μL of 6 M KCl, and 0.1% Tween 20 were added to the sample reservoir. Next 200 μg of the magnetic silica beads in 2 μL water were added, mixed well, and incubated for 15 minutes. Subsequently, the silica beads were magnetically dragged into Channel 1. Once the silica beads carrying the protein-bound miRNAs left the sample reservoir, 60 μL of 6 M guanidine thiocyanate, 1 μL of 10% tween 20, 15 μL of 100% ethanol and 200 μg of silica beads were added, mixed well, and incubated for 15 minutes. This time the beads would extract the lipoprotein-bound miRNAs and be moved to Channel 2. In both Channel 1 and 2, the silica beads were moved through the wash and elution reservoirs, and eventually collected in the corresponding bead reservoir.

The three eluents corresponding to the exosome, protein, and lipoprotein-bound miRNA fractions were removed from the chip, and quantified by RT-qPCR with the commercial Taqman miRNA primer assay kits specific to each target miRNA.

Confirmation of Exosome Isolation and Disruption to Release the Exosomal miRNAs.

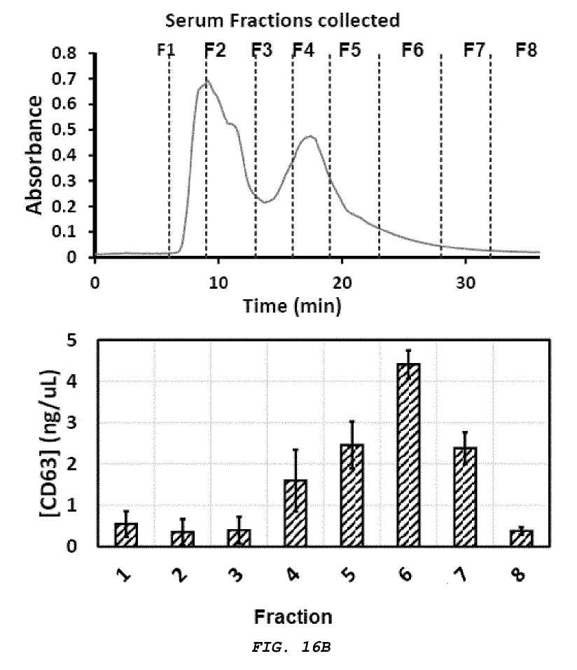

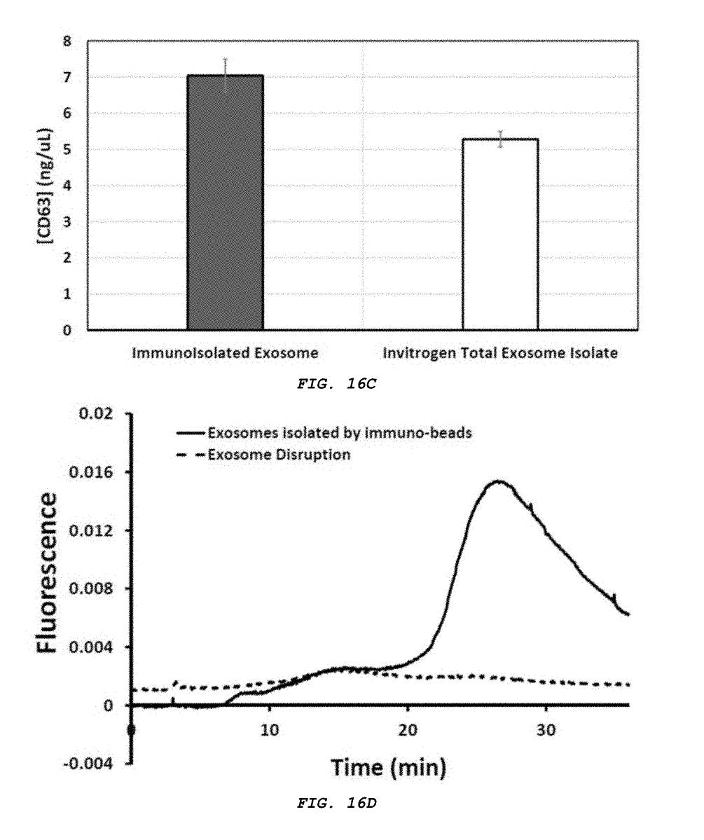

Extraction of exosomes was confirmed by analyzing the extracted samples with asymmetrical flow field flow fractionation (AF4) and comparison of the CD63 amounts in the microfluidic-chip extraction and in the exosomes prepared by the Invitrogen Exosome Isolation Kit. AF4 separates analytes based on their hydration sizes. All samples were examined by UV-Vis absorption; and also stained with the DiO dye and detected by fluorescence for illustration of the lipid-enriched portions. As shown in FIG. 16a, both samples prepared by the on-chip immuno-extraction and by the Invitrogen kit showed significant peaks at elution time later than 20 min, within which CD63 was detected at significant amounts by ELISA in the eluents (FIG. 16b). The sample prepared by the Invitrogen kit also had a relatively small peak eluted between 10-17 min, which could be the lipoprotein structures that were co-precipitated during centrifugation. The exosome peak in the sample isolated by the immune-beads showed up at a later time than the one prepared by the Invitrogen kit. The CD63 concentration found in the method of the disclosure was 7.04 ng/μL (FIG. 16c), matching well with the sum CD63 concentration found in Fraction 6-8; and that with the Invitrogen kit was 5.28 ng/μL, agreeing with the sum CD63 in Fraction 4-5. The present method yielded a higher recovery for exosomes from serum than the Invitrogen kit, and a relatively larger exosome fraction. From FIG. 16b, one can also note that there were large exosomes eluted in Fraction 7, which was not collected in the AF4 method.

Once the exosomes were isolated, they were treated with a solution containing 75% EtOH. The high organic content destroyed the membrane structure of the exosomes, and released the enclosed miRNAs. FIG. 16d show that, after the treatment, the exosome peak disappeared when analyzed by AF4. Since the DiO dye emits strong fluorescence only in a hydrophobic environment, the dramatic decrease in the fluorescence signal indicates the lack of intact hydrophobic structure and the success of exosome disruption.

Confirmation of Disruption of the miRNA-Protein Complexes.

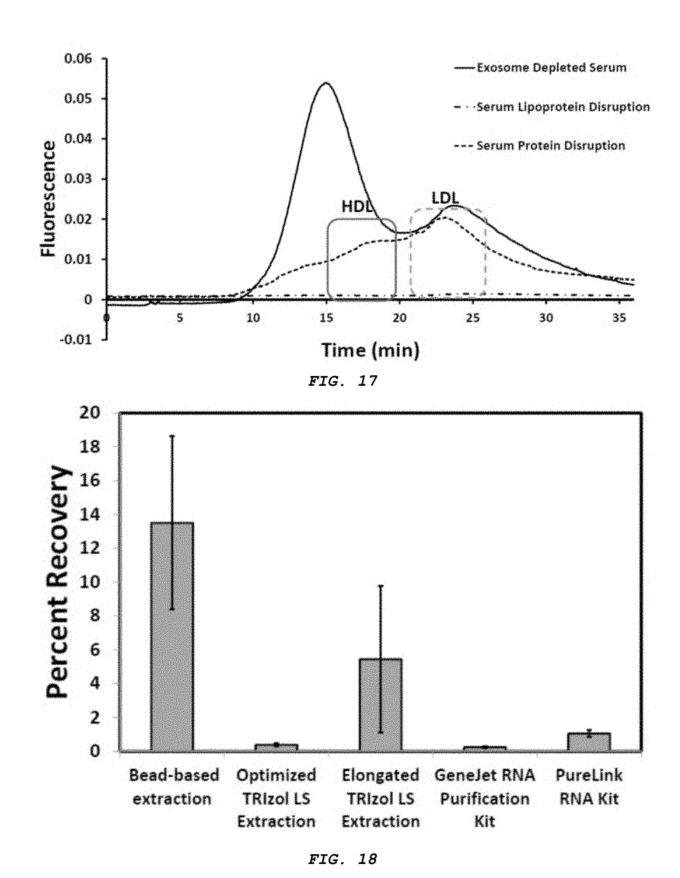

Once the exosomes were depleted, the remaining miRNAs in the sample were bound to lipoprotein complexes or to proteins such as AGO2. Protein-RNA interaction relies on H-bonding and electrostatic interaction between the negatively charged phosphate groups on RNA and the positively charged primary amines on proteins. The presence of denaturants for both RNAs and proteins would definitely affect the stability of the protein-RNA complexes, releasing the protein-bound miRNAs. To disrupt the more compact lipoprotein complexes, higher concentrations or stronger denaturants should be employed. The denaturants chosen were the combination of two Chaotropic salts, Guanidine HCl (GuHCl) and Guanidine Thiocyanate (GuSCN), the surfactant Tween 20, and the organic solvent EtOH. To realize consecutive extraction of the protein- and lipoprotein-bound miRNAs from the same serum sample, the serum depleted of exosomes was treated using the mild solution that contained about 0.5 M KCl, 0.0015% Tween 20, and 4 M Guanidine HCl to release the miRNAs bound to proteins. Once these miRNAs were removed by silica beads, more Tween 20 and the stronger denaturants of Guanidine Thiocyanate (GuSCN) and EtOH were supplied to break up the compact structures of the lipoprotein complexes and free the associated miRNAs. The final mixture contained roughly 0.25 M KCl, 1.8 M GuHCl, 2.5 M GuSCN, and 10% EtOH. Again, DiO staining and AF4 analysis was used to visualize the integrity of the HDL and LDL complexes. Before any treatment, serum stained with the DiO dye gave two large peaks when injected into the AF4 system (FIG. 17). Once treated with the mild denaturing solution, the fluorescence intensity of the first peak dropped significantly. This peak represented the elution of immunoglobulins as proved in the preliminary study, which was stained by DiO as well. The loss of its fluorescence indicates that its folding was interrupted, meeting the expectation for the function of the mild denaturing solution. Moreover, after the serum was incubated with the stronger denaturing solution that contained GuSCN and EtOH, the trace became flat and no distinct fluorescent peak was detected in the fractogram (FIG. 17), pointing out the disruption of the lipoprotein structures.

Extraction of Free miRNAs by Silica Beads.

Disruption of exosomes, lipoprotein complexes, and simple protein-RNA complexes release the miRNAs from their carriers. The freed miRNAs then bind to the silica beads. Silica-based DNA or RNA extraction has been widely employed. Typically, chaotropic salts like GuHCl would be used to weaken the hydration effect of nucleic acids in aqueous solutions and promote hydrophobic interaction with the silica surface. KCl could also be included to enhance binding by forming salt bridges between the ionized silanol groups on silica surface and the phosphate backbone of RNA. Both were either present in the mild denaturing solution for protein-RNA disruption; or added with the silica beads after exosome disruption. Any extraction procedure could experience sample loss due to binding equilibrium and diffusion. To evaluate the recovery of in the microfluidic-chip method, total miRNA extraction using the Trizol reagent and two other commercial kits, the GeneJet Kit (Thermo Scientific Catalog# K0731) and PureLink Kit (Ambion, Catalog#12183020) were used. These represent the common methods employed to extract RNAs from biological samples. Following the manufacturer's protocols, only a very small fraction of the cel-miR-67 spiked in the serum was recovered using the commercial kits (FIG. 18). This could be due to short length of the miRNAs compared to mRNA and genomic DNA that does not provide large interaction surface with the particles. The on-chip extraction method led to the highest average recovery of 13.5%, which could be attributed to its minimal manual handling and liquid transfer compared to the commercial kits or reagents.

Fractionation Effect Evaluation.

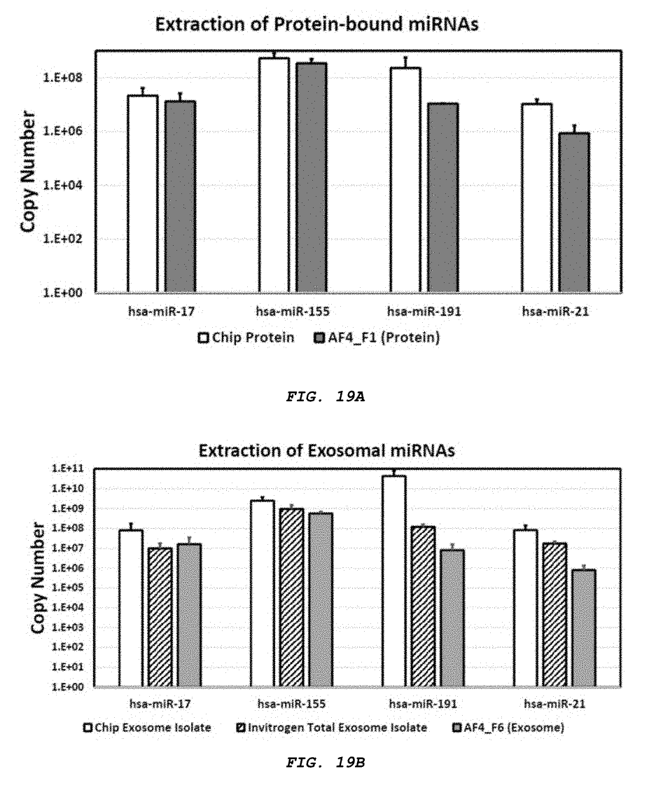

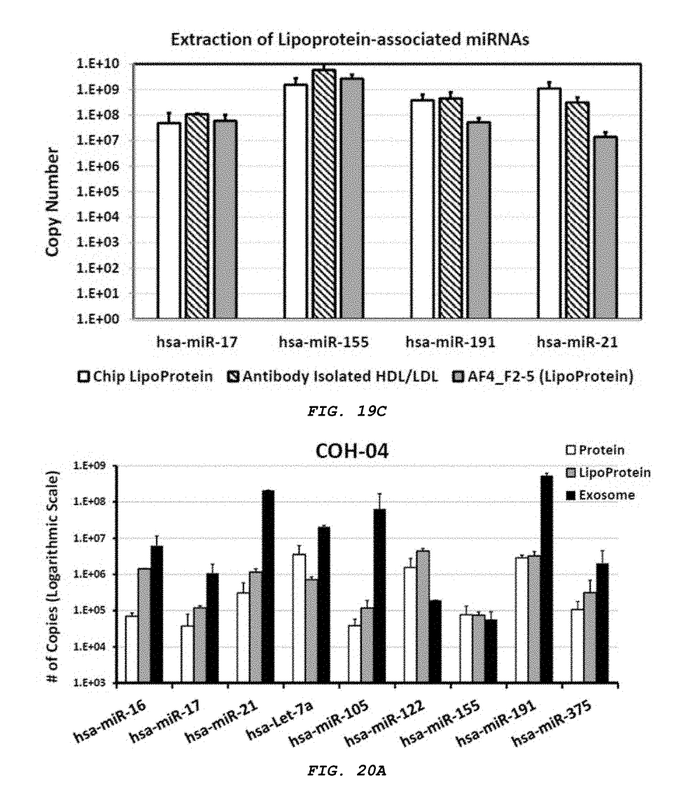

The above results prove that, the fractionation method does not induce more sample loss and takes much less time than the conventional RNA extraction methods, while separately obtain the miRNAs bound to three different carriers. To further evaluate the fractionation effect, the miRNA amounts recovered from on-chip extraction were compared with those obtained from the AF4 method. Fraction 1 and Fraction 6 obtained by the AF4 method represented the protein-bound (grey bars in FIG. 19a) and exosomal miRNAs (grey bars in FIG. 19b), respectively; and their amounts were compared with those obtained with the microchip method in Channel 1 and 3 (white bars in FIGS. 19a and c). The Invitrogen Total Exosome Isolation kit was also employed to obtain exosomes, and the exosomal miRNAs were attained by TRIzol extraction (patterned bar in FIG. 19b). The miRNA amounts recovered from Fraction 2-5 with the AF4 method were considered the lipoprotein-bound miRNAs and compared with the miRNAs extracted in Channel 2 (FIG. 19c). Finally, the HDL/LDL complexes that were isolated by the immuno-beads conjugated with the anti-HDL/LDL IgGs, and miRNAs were extracted by TRIzol and added to the comparison. Four miRNAs were tested: miR-17, -21, -155, and -191.

As shows in FIG. 19 the on-chip extraction method recovered comparable or higher amounts of miRNAs from each type of the carrier than the AF4 method. Separation by AF4 produces large elution volume up to several mLs, making the subsequent miRNA recovery with TRIzol more difficult and time consuming (>1 day) and with lower yield. In contrast, the on-chip extraction starts with 25 μL serum and the final volume did not exceed 150 μL and the method can be completed within 1.5 hrs. As for the exosomal miRNAs, Fraction 7 indicated in FIG. 16a was not collected in the present design of the AF4 protocol, and the miRNAs enclosed in the larger exosomes eluted in this fraction were not extracted. All these contribute to the higher miRNA recovery with the microchip method. On-chip extraction of the exosomal miRNAs yielded higher amounts of miRNAs than the Invitrogen Total Exosome Isolation kit (white vs. patterned bars in FIG. 19b). This may be due to the method of the disclosure being capable of recovering more of the large exosomes with less contamination from the lipoprotein complexes, as shown in FIG. 16a when analyzing the exosome size distribution and purity using AF4. Interestingly, comparable miRNA amounts were obtained for the lipoprotein-associated miRNAs using the microchip method and via immuno-capture (white vs. patterned bars in FIG. 19c). Without using the costly antibody and complicated affinity capture, the lipoprotein-bound miRNAs were isolated by various chemicals, a surprisingly simple but effective approach, and yielded comparable, if not higher, recovery for all four miRNA strands tested.

Analysis of Human Sera.

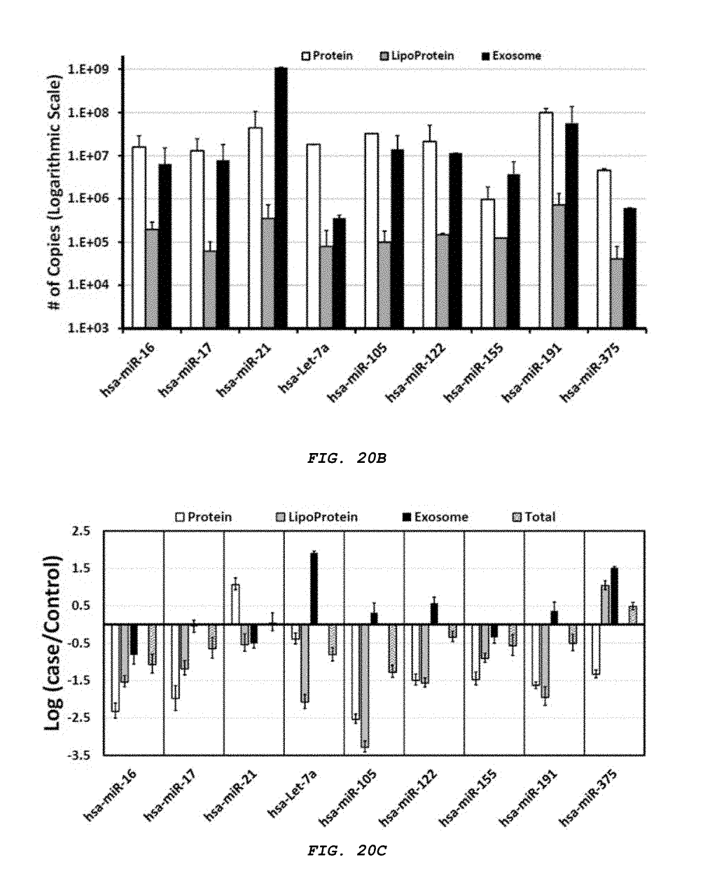

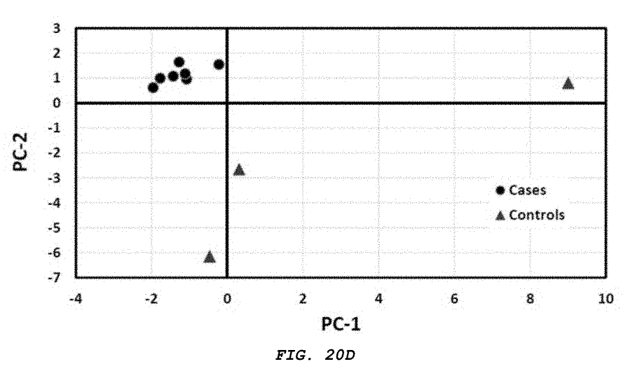

7 sera samples from human subject were obtained. These samples were collected from breast cancer patients. In addition, healthy sera was purchased from Innovative Research Inc., matching the age and race of each of the cancer subjects, as the controls. The miRNA distribution profiles of one case and one control are provided in FIGS. 20a and b. As can be seen each miRNA shows distinct pattern in its distribution among the three main carriers, which are related to how they are secreted and transported. By comparing the average miRNA content in each fraction from all (n=7) cases with that from all (n=3) controls, larger changes in individual fractions were identified compared to using the changes in total miRNA content (FIG. 20c). The patterned bars in FIG. 20c represent the change in total miRNA content between the cases and the controls. These changes were typically within the range of ±0.5, indicating the fold change in miRNA content was smaller than 10, which is considered not reliable in large scale analysis of miRNA expression profiles in human samples because of the large differences between individuals. In contrast, most of the changes in the fractionated miRNA levels are beyond the range of ±1. For instant, the protein-bound miR-16 and -155 levels in patient samples were more than 100 fold lower than those found in the healthy individuals. The lipoprotein-associated miR-105 level was even more than 1000 fold lower in cancer patients. Close to 100 fold increase in the exosomal Let-7a and miR-375 contents were identified in the cases compared to the controls. Interestingly, subjecting the distribution profiles of all samples to Principle Component Analysis (PCA) yielded clear grouping between all cases and all controls on the scatter plot obtained from the first two principle components (FIG. 20d). These two principle component summarized over 78% of the overall variance of the data set. In contrast, using the total miRNA contents or the exosomal miRNA fraction, no clear separation between the cases and controls was observed after PCA. These results well prove that distribution profiling can reveal larger changes between cancer patients and healthy individuals, compared to the conventional methods.

A number of embodiments have been described herein. Nevertheless, it will be understood that various modifications may be made without departing from the spirit and scope of this disclosure. Accordingly, other embodiments are within the scope of the following claims.

1. A fractionation method for determining the distribution of circulating RNAs in a sample, comprising:
fractionating a biological fluid sample obtained from a subject into fractions comprising at least an exosome fraction, protein fraction and lipoprotein fraction, wherein each fraction comprises RNA carriers;
determining or quantitating the RNAs in each of the fractions to generate a distribution profile for the RNAs to RNA carriers in the sample.

2. The method of claim 1, wherein the fractionating is (a) by performing asymmetrical flow field-flow fractionation (AF4) on the sample and collecting a plurality of eluents or (b) by a chip-based microfluidics system.

3. (canceled)

4. The method of claim 1, wherein the biological fluid sample is a serum sample.

5. The method of claim 2, wherein a serum sample is fractionated using a trapezoidal separation channel about 0.350 mm in thickness and a tip-to-tip length of about 275 mm, with an inlet triangle width of about 20 mm and outlet width of about 5 mm.

6. The method of claim 5, wherein the surface area of the accumulation wall is about 3160 mm² with a molecular weight cutoff value of 10 kDA.

7. The method of claim 2, wherein the plurality of eluents are collected as 1 minute eluents over a period of 20 to 25 minutes.

8. The method of claim 2, wherein at least six fractions of the biological fluid sample are generated from the plurality of eluents.

9. The method of claim 8, wherein the six fractions result from combining 1 minute eluents collected over six separate and non-overlapping time periods and wherein each of the six factions are enriched with an RNA carrier protein of a specific hydrodynamic diameter.

10. (canceled)

11. The method of claim 1, wherein the fractions are enriched with proteins, high density lipoprotein (HDL), low density lipoprotein (LDL) and exosome.

12. The method of claim 1, wherein the RNAs are determined or quantified by deep sequencing or RT-qPCR.

13. The method of claim 1, wherein the RNAs include microRNAs or IncRNAs, or viral RNAs.

14. The method of claim 13, wherein the microRNAs or IncRNAs or viral RNAs are biomarkers associated with a disease or disorder.

15. The method of claim 14, wherein the disorder is cancer.

16. The method of claim 15, wherein the cancer is breast cancer.

17. The method of claim 1, wherein the set of RNAs are microRNAs comprising the sequence of SEQ ID NO:1, 2, 3, 4, 5, 6, 7, 8, and/or 9.

18. The method of claim 1, wherein the set of RNAs is selected from the group consisting of let-7a, let-7b, let-7c, let-7d, let-7e, let-7f, let-7g, let-7i, miR-1, miR-100, miR-101, miR-103, miR-105, miR-106a, miR-106b, miR-107, miR-10a, miR-10b, miR-122a, miR-124a, miR-125a, miR-125b, miR-126, miR-126*, miR-127, miR-128a, miR-128b, miR-129, miR-130a, miR-130b, miR-132, miR-133a, miR-133b, miR-134, miR-135a, miR-135b, miR-136, miR-137, miR-138, miR-139, miR-140, miR-141, miR-142-3p, miR-142-5p, miR-143, miR-144, miR-145, miR-146a, miR-146b, miR-147, miR-148a, miR-148b, miR-149, miR-150, miR-151, miR-152, miR-153, miR-154, miR-154*, miR-155, miR-15a, miR-15b, miR-16, miR-17-3p, miR-17-5p, miR-181a, miR-181b, miR-181c, miR-181d, miR-182, miR-182*, miR-183, miR-184, miR-185, miR-186, miR-187, miR-188, miR-189, miR-18a, miR-18a*, miR-18b, miR-190, miR-191, miR-191*, miR-192, miR-193a, miR-193b, miR-194, miR-195, miR-196a, miR-196b, miR-197, miR-198, miR-199a, miR-199a*, miR-199b, miR-19a, miR-19b, miR-200a, miR-200a*, miR-200b, miR-200c, miR-202, miR-202*, miR-203, miR-204, miR-205, miR-206, miR-208, miR-20a, miR-20b, miR-21, miR-210, miR-211, miR-212, miR-213, miR-214, miR-215, miR-216, miR-217, miR-218, miR-219, miR-22, miR-220, miR-221, miR-222, miR-223, miR-224, miR-23a, miR-23b, miR-24, miR-25, miR-26a, miR-26b, miR-27a, miR-27b, miR-28, miR-296, miR-299-3p, miR-299-5p, miR-29a, miR-29b, miR-29c, miR-301, miR-302a, miR-302a*, miR-302b, miR-302b*, miR-302c, miR-302c*, miR-302d, miR-30a-3p, miR-30a-5p, miR-30b, miR-30c, miR-30d, miR-30e-3p, miR-30e-5p, miR-31, miR-32, miR-320, miR-323, miR-324-3p, miR-324-5p, miR-325, miR-326, miR-328, miR-329, miR-33, miR-330, miR-331, miR-335, miR-337, miR-338, miR-339, miR-33b, miR-340, miR-342, miR-345, miR-346, miR-34a, miR-34b, miR-34c, miR-361, miR-362, miR-363, miR-363*, miR-365, miR-367, miR-368, miR-369-3p, miR-369-5p, miR-370, miR-371, miR-372, miR-373, miR-373*, miR-374, miR-375, miR-376a, miR-376a*, miR-376b, miR-377, miR-378, miR-379, miR-380-3p, miR-380-5p, miR-381, miR-382, miR-383, miR-384, miR-409-3p, miR-409-5p, miR-410, miR-411, miR-412, miR-421, miR-422a, miR-422b, miR-423, miR-424, miR-425, miR-425-5p, miR-429, miR-431, miR-432, miR-432*, miR-433, miR-448, miR-449, miR-450, miR-451, miR-452, miR-452*, miR-453, miR-455, miR-483, miR-484, miR-485-3p, miR-485-5p, miR-486, miR-487a, miR-487b, miR-488, miR-489, miR-490, miR-491, miR-492, miR-493, miR-493-3p, miR-494, miR-495, miR-496, miR-497, miR-498, miR-499, miR-500, miR-501, miR-502, miR-503, miR-504, miR-505, miR-506, miR-507, miR-508, miR-509, miR-510, miR-511, miR-512-3p, miR-512-5p, miR-513, miR-514, miR-515-3p, miR-515-5p, miR-516-3p, miR-516-5p, miR-517*, miR-517a, miR-517b, miR-517c, miR-518a, miR-518a-2*, miR-518b, miR-518c, miR-518c*, miR-518d, miR-518e, miR-518f, miR-518f*, miR-519a, miR-519b, miR-519c, miR-519d, miR-519e, miR-519e*, miR-520a, miR-520a*, miR-520b, miR-520c, miR-520d, miR-520d*, miR-520e, miR-520f, miR-520g, miR-520h, miR-521, miR-522, miR-523, miR-524, miR-524*, miR-525, miR-525*, miR-526a, miR-526b, miR-526b*, miR-526c, miR-527, miR-532, miR-542-3p, miR-542-5p, miR-544, miR-545, miR-548a, miR-548b, miR-548c, miR-548d, miR-549, miR-550, miR-551a, miR-552, miR-553, miR-554, miR-555, miR-556, miR-557, miR-558, miR-559, miR-560, miR-561, miR-562, miR-563, miR-564, miR-565, miR-566, miR-567, miR-568, miR-569, miR-570, miR-571, miR-572, miR-573, miR-574, miR-575, miR-576, miR-577, miR-578, miR-579, miR-580, miR-581, miR-582, miR-583, miR-584, miR-585, miR-586, miR-587, miR-588, miR-589, miR-590, miR-591, miR-592, miR-593, miR-594, miR-595, miR-596, miR-597, miR-598, miR-599, miR-600, miR-601, miR-602, miR-603, miR-604, miR-605, miR-606, miR-607, miR-608, miR-609, miR-610, miR-611, miR-612, miR-613, miR-614, miR-615, miR-616, miR-617, miR-618, miR-619, miR-620, miR-621, miR-622, miR-623, miR-624, miR-625, miR-626, miR-627, miR-628, miR-629, miR-630, miR-631, miR-632, miR-633, miR-634, miR-635, miR-636, miR-637, miR-638, miR-639, miR-640, miR-641, miR-642, miR-643, miR-644, miR-645, miR-646, miR-647, miR-648, miR-649, miR-650, miR-651, miR-652, miR-653, miR-654, miR-655, miR-656, miR-657, miR-658, miR-659, miR-660, miR-661, miR-662, miR-663, miR-7, miR-9, miR-9*, miR-92, miR-93, miR-95, miR-96, miR-98, miR-99a, miR-99b and any combination thereof.

19. The method of claim 2, wherein the chip-based microfluidic system comprises:
a microfluidic chip comprising
at least 3 channels;
at least 3 reservoirs; and
a sample reservoir,
wherein the channels fluidly connect the at least 3 reservoirs and sample reservoir;
a first bead reagent comprising magnetic beads and an antibody that interacts with an antigen on exosomes; and
a second bead reagent comprising cationically charged beads.

20. The method of claim 19, wherein the antibody is an anti-CD63 antibody.

21. The method of claim 19, wherein the method comprises
(i) adding serum to the sample reservoir;
(a) adding the first bead reagent to the sample reservoir; applying a magnetic field to the sample reservoir and moving the first bead reagent with the magnetic field through a first channel of the at least 3 channels to a first reservoir of the at least 3 reservoirs; disrupting the exosomes in the first reservoir; removing the first bead reagent; adding a second bead reagent to the first reservoir;
(b) adding GuHCl, KCl, and a detergent to the sample reservoir to dissociate RNA from proteins; add the second bead reagent to the sample reservoir to bind RNA; moving the second bead reagent through a second channel of the at least 3 channels to a second reservoir of the at least 3 reservoirs; and
(c) adding guanidine thiocyanate, a detergent, and ethanol to the sample reservoir to dissociate RNA from lipoproteins; add the second bead reagent to the sample reservoir to bind RNA; moving the second bead reagent through a third channel of the at least 3 channels to a third reservoir of the at least 3 reservoirs,
(ii) extracting RNA from each of the first, second and third reservoir.

22. The method of claim 19, further comprising reagents that can destroy the protein-RNA interaction, or the lipoprotein complexes.

23. The method of claim 22, wherein the reagents are a mixture of surfactant, organic solvent, chaotropic salts.

24. A method for diagnosing whether a subject has a disorder, comprising:
comparing the distribution of circulating RNAs obtained by using the method of claim 1 between a healthy subject(s) and subject(s) with the disorder, wherein a difference identifies a risk of the disease or disorder.

25. A kit for carrying out any of the methods of claim 1, wherein the kit is compartmentalized to contain reagents and devices for performing the methods.

26. The kit of claim 25 comprising a microfluidic device, a first bead reagent, a second bead reagent, and reagents that can destroy the protein-RNA interaction, or can destroy the lipoprotein complexes.


SEQUENCE LISTING (9 sequences)

SEQ ID NO: 1 - RNA, 23 nt, Artificial Sequence
  cel-MiR-67 Internal Standard
cgcucauucugccgguuguuaug

SEQ ID NO: 2 - RNA, 22 nt, homo sapiens
ugagguaguagguuguauaguu

SEQ ID NO: 3 - RNA, 22 nt, Homo sapiens
uagcagcacguaaauauuggcg

SEQ ID NO: 4 - RNA, 23 nt, Homo sapiens
caacggaaucccaaaagcagcug

SEQ ID NO: 5 - RNA, 23 nt, Homo sapiens
caaagugcuuacagugcagguag

SEQ ID NO: 6 - RNA, 23 nt, Homo sapiens
uuaaugcuaaucgugauaggggu

SEQ ID NO: 7 - RNA, 22 nt, Homo sapiens
uuuguucguucggcucgcguga

SEQ ID NO: 8 - RNA, 22 nt, Homo sapiens
uagcuuaucagacugauguuga

SEQ ID NO: 9 - RNA, 22 nt, Homo sapiens
uggagugugacaaugguguuug